(19) United States
(12) Patent Application Publication
(10) Pub. No.: US 2023/0234046 A1
(43) Pub. Date: Jul. 27, 2023

(54) AUTOMATIC MULTI-STEP REACTION DEVICE

(71) Applicant: President and Fellows of Harvard College, Cambridge, MA (US)

(72) Inventors: Thomas E. Schaus, Cambridge, MA (US); Nikhil Gopalkrishnan, Cambridge, MA (US); Peng Yin, Cambridge, MA (US)

(21) Appl. No.: 18/002,629

(22) PCT Filed: Jun. 24, 2021
(86) PCT No.: PCT/US21/38930

§ 371 Date: Dec. 20, 2022

Related U.S. Application Data
(60) Provisional application No. 63/043,232, filed on Jun. 24, 2020.
(60) Provisional application No. 63/046,424, filed on Jun. 30, 2020.
(60) Provisional application No. 63/082,776, filed on Sep. 24, 2020.
(60) Provisional application No. 63/083,640, filed on Sep. 25, 2020.

(51) Int. Cl. B01L 3/00; G01N 33/543

(52) U.S. Cl. CPC B01L 3/5023; G01N 33/54388; B01L 3/502715; B01L 3/5029; B01L 3/50825; B01L 2200/16; B01L 2300/042; B01L 2400/0487; B01L 2300/0877; B01L 2300/0825; B01L 2200/0689; B01L 2300/18

(57) ABSTRACT

A device for performing an assay comprises a tube, a cap, an insert, and a reaction container. The tube includes a lateral flow strip disposed therein. The cap is coupled to the tube and includes a hollow interior defined at least partially therethrough. The insert is configured to be at least partially received within the hollow interior of the cap. The reaction container includes a cavity configured to store one or more fluids therein, and is rotatably coupled to the cap such that rotation of the cap relative to the reaction container causes (i) mixing of the one or more fluids and (ii) at least a portion of the mixed fluids to be delivered from the reaction container to the lateral flow strip via the insert.

### CROSS-REFERENCE TO RELATED APPLICATIONS

This application claims the benefit of, and priority to, U.S. Provisional Patent Application No. 63/083,640, filed on Sep. 25, 2020; U.S. Provisional Patent Application No. 63/082,776, filed on Sep. 24, 2020; U.S. Provisional Patent Application No. 63/046,424, filed on Jun. 30, 2020; and U.S. Provisional Patent Application No. 63/043,232, filed on Jun. 24, 2020, each of which is hereby incorporated by reference herein in its entirety.

### GOVERNMENT SUPPORT

This invention was made with U.S. Government support under grant nos. 1DP1GM133052-01, 5DP1GM133052-02, and 1R21CA235421-01 awarded by the National Institutes of Health. The U.S. Government has certain rights in the invention.

### TECHNICAL FIELD

The technology described herein relates to a set of mechanisms that enable biochemical reactions within a closed container, e.g., a tube, using a screw turn mechanism or other mechanism to allow a user to easily and reliably advance the reaction in a manual, stepwise process.

### BACKGROUND

In some reactions, for example those in which DNA is amplified (e.g., copied exponentially), it is useful to keep the products of the reaction enclosed. In the case of highly sensitive amplification of specific nucleic acid targets, as is increasingly common in the diagnosis of SARS-CoV-2 and other infectious disease with Nucleic Acid Amplification Tests (NAATs), any amplification product released resembles the template (target) itself, and so can contaminate future tests and yield false positives. In other applications, the final mixture may be toxic or sensitive to surrounding chemicals or the environment. Moreover, it is common in modern usage to use very small volumes in biochemical reactions, including 50 μl, 10 μl, or less, where surface tension, viscosity, and hydrophobic/hydrophilic forces are high compared to mass effects such as weight and inertia. Further, the ratio of inertial forces to viscous forces are generally low, causing mixing processes to be laminar and often incomplete. Thus, there is a need in the art for devices, reaction vessels and/or containers that provide reliable reactions while controlling for operational factors that could cause testing errors.

### SUMMARY

According to one implementation of the present disclosure, a device for performing a multi-step assay comprises a tube, a cap, an insert, and a reaction container. The tube includes a lateral flow strip disposed therein. The cap is coupled to the tube and includes a hollow interior defined therethrough. The insert is configured to be partially received within the hollow interior of the cap. The reaction container includes a cavity configured to store one or more fluids therein. The reaction container is rotatably coupled to the cap, such that rotation of the cap relative to the reaction container causes (i) the one or more fluids to be mixed, and (ii) at least a portion of the mixed fluids to be delivered from the reaction container to the lateral flow strip, via the insert.

In some aspects of the implementation, the reaction container includes a first well storing a first reagent, a second well storing a second reagent, a third well storing a buffer, and a seal covering an opening of the third well. In some aspects of the implementation, the insert includes a body, a displacing bump extending from the body, a brush extending from the body and an aperture defined through the body. In response to the reaction container being rotated relative to the cap to a first position, the brush aids in mixing the first reagent stored in the first well and the second reagent stored in the second well. In response to the reaction container being rotated relative to the cap from the first position to the second position, the displacing bump is configured to break the seal of the third well to mix the buffer with the mixed first reagent and second reagent. In response to the reaction container being rotated relative to the cap from the second position to the third position, the aperture of the body is configured to deliver the mixed first reagent, second reagent, and buffer from the reaction chamber to the lateral flow strip.

In some aspects of the implementation, the reaction container is configured to store a first reagent, and the insert includes a blister pack configured to store a buffer. The reaction container includes a protrusion configured to engage the blister pack and cause mixing of the first reagent and the buffer in response to the reaction container being rotated relative to the cap toward a first position.

According to another implementation of the present disclosure, a device for performing a multi-step assay comprises a cap, a lateral flow strip, a plunger assembly, a reagent insert, and a reaction container. The cap includes a hollow interior defined therethrough. The plunger assembly includes a primary plunger and a secondary plunger, and is configured to be received within the hollow interior of the cap. The reagent insert includes a primary aperture, a secondary aperture, a slot, and a seal. The primary aperture is configured to store a first reagent. The secondary aperture is configured to store a second reagent. The slot is configured to receive a portion of the lateral flow strip therein. The seal is positioned such that it covers an end of both the primary aperture and the secondary aperture. The reaction container includes an internal cavity configured to store a buffer and receive a portion of the reagent insert therein. In response to rotation of the reaction container relative to the cap to a first position, the primary plunger pierces the seal to mix the first reagent and the buffer. In response to rotation of the reaction container relative to the cap from the first position to a second position, the secondary plunger pierces the seal to mix the second reagent with the mixed first reagent and buffer. In response to rotation of the reaction container relative to the cap from the second position to the third position, the mixed first reagent, second reagent, and buffer are transported from the reaction container to the lateral flow strip through the reagent insert.

According to a further implementation of the present disclosure, a device for performing one or more tests on one or more samples comprises a collection assembly and a reaction container. The collection assembly includes a handle and a plurality of collection swabs extending from the handle. The reaction container includes a plurality of reaction chambers. Each of the plurality of reaction chambers is associated with a corresponding one of the plurality of collection swabs. In response to a configuration of the device moving from an unassembled configuration to an assembled configuration, the collection assembly is coupled to the reaction container, and each of the plurality of reaction chambers at least partially houses the corresponding one of the plurality of collection swabs therein.

Additional aspects of the present disclosure will be apparent to those of ordinary skill in the art in view of the detailed description of various implementations, which is made with reference to the drawings, a brief description of which is provided below.

### BRIEF DESCRIPTION OF THE DRAWINGS

Features and advantages of the present disclosure will become more apparent from the following detailed description of exemplary implementations thereof taken in conjunction with the accompanying drawings in which:

FIG. 1A illustrates a first device for performing an assay, according to some implementations of the present disclosure;

FIG. 1B illustrates the device of FIG. 1A when assembled for use, according to implementations of the present disclosure;

FIG. 2A illustrates a first step of an assay using the device of FIG. 1A, according to some implementations of the present disclosure;

FIG. 2B illustrates a second step of an assay using the device of FIG. 1A, according to some implementations of the present disclosure;

FIG. 2C illustrates a third step of an assay using the device of FIG. 1A, according to some implementations of the present disclosure;

FIG. 2D illustrates a fourth step of an assay using the device of FIG. 1A, according to some implementations of the present disclosure;

FIG. 2E illustrates a fifth step of an assay using the device of FIG. 1A, according to some implementations of the present disclosure;

FIG. 3A illustrates a cross-sectional side view of the device of FIG. 1A when all components are coupled together, according to some implementations of the present disclosure;

FIG. 3B illustrates a cross-sectional perspective view of the device of FIG. 1A when all components are coupled together, according to some implementations of the present disclosure;

FIG. 3C illustrates an exploded perspective view of the device of FIG. 1A when all components are coupled together, according to some implementations of the present disclosure;

FIG. 3D illustrates a perspective view of the device of FIG. 1A when all components are coupled together, according to some implementations of the present disclosure;

FIG. 4A illustrates a second device for performing an assay, according to some implementations of the present disclosure;

FIG. 4B illustrates the device if FIG. 4A when assembled for use, according to some implementations of the present disclosure;

FIG. 5A illustrates a first step of an assay using the device of FIG. 4A, according to some implementations of the present disclosure;

FIG. 5B illustrates a second step of an assay using the device of FIG. 4A, according to some implementations of the present disclosure;

FIG. 5C illustrates a third step of an assay using the device of FIG. 4A, according to some implementations of the present disclosure;

FIG. 5D illustrates a fourth step of an assay using the device of FIG. 4A, according to some implementations of the present disclosure;

FIG. 6A illustrates a third device for performing an assay, according to some implementations of the present disclosure;

FIG. 6B illustrates a top view of a reagent insert of the device of FIG. 6A, according to some implementations of the present disclosure;

FIG. 6C illustrates the device of FIG. 6A when assembled for use, according to some implementations of the present disclosure;

FIG. 7A illustrates a first step of an assay using the device of FIG. 6A, according to some implementations of the present disclosure;

FIG. 7B illustrates a second step of an assay using the device of FIG. 6A, according to some implementations of the present disclosure;

FIG. 7C illustrates a third step of an assay using the device of FIG. 6A, according to some implementations of the present disclosure;

FIG. 7D illustrates a fourth step of an assay using the device of FIG. 6A, according to some implementations of the present disclosure;

FIG. 7E illustrates a fifth step of an assay using the device of FIG. 6A, according to some implementations of the present disclosure;

FIG. 8A illustrates a partial cross-sectional perspective view of a fourth device for performing an assay in an unassembled configuration, according to some implementations of the present disclosure;

FIG. 8B illustrates a partial cross-sectional perspective view of the device of FIG. 8A in an assembled configuration, according to some implementations of the present disclosure;

FIG. 9A illustrates a top perspective view of a fifth device for performing an assay in an unassembled configuration, according to some implementations of the present disclosure;

FIG. 9B illustrates a cross-sectional perspective view of the device of FIG. 9A in an assembled configuration, according to some implementations of the present disclosure;

FIG. 9C illustrates a cross-sectional top view of the device of FIG. 9A in the assembled configuration, according to some implementations of the present disclosure;

FIG. 10A illustrates a first heating block for controlling the temperature during an assay, according to some implementations of the present disclosure;

FIG. 10B illustrates a second heating block for controlling the temperature during an assay, according to some implementations of the present disclosure;

FIG. 10C illustrates a third heating block for controlling the temperature during an assay, according to some implementations of the present disclosure;

FIG. 11A illustrates a first timing mechanism for tracking time during an assay, according to some implementations of the present disclosure;

FIG. 11B illustrates a second timing mechanism for tracking time during an assay, according to some implementations of the present disclosure;

FIG. 12A illustrates a first mechanism for advancing a reaction container during an assay, according to some implementations of the present disclosure;

FIG. 12B illustrates a second mechanism for advancing a reaction container during an assay, according to some implementations of the present disclosure; and

FIG. 12C illustrates a third mechanism for advancing a reaction container during an assay, according to some implementations of the present disclosure.

While the present disclosure is susceptible to various modifications and alternative forms, specific implementations have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

While this invention is susceptible of embodiment in many different forms, there is shown in the drawings and will herein be described in detail preferred aspects of the invention with the understanding that the present disclosure is to be considered as an exemplification of the principles of the invention and is not intended to limit the broad aspect of the invention to the aspects illustrated. For purposes of the present detailed description, the singular includes the plural and vice versa (unless specifically disclaimed); the words “and” and “or” shall be both conjunctive and disjunctive; the word “all” means “any and all”; the word “any” means “any and all”; and the word “including” means “including without limitation.”

In some multi-step reactions, it is desirable that stepwise reactions be easily controlled by untrained or lightly trained (non-expert, non-professional) users, ranging from healthcare workers operating point of care tests to consumers testing at home, and so should be straightforward and easy to control. In some implementations, reactant amounts may be pre-metered and there would be no need for precise operation on the part of the user. This contrasts with the pipetting of small volumes with user-calibrated equipment, with which errors are frequently made. A desirable product may include precise operation internally with only straightforward, coarse treatment by the user. It is desirable for reactions including the movement of small volumes of reagents to be driven by mechanisms that provide precise volume movement, timing, and mixing.

Provided herein are mechanisms and devices that enable reliable, consistent multi-step reactions within closed reaction containers, moving and mixing volumes of 10-200 μl with a simple rotation mechanism. Implementations of the various aspects described herein can be used for diagnostic purposes, such as performing amplification reactions to detect a target where the problems of contamination with amplicons and ease of use are critical. Amplification reactions that can be performed can include a polymerase chain reaction (PCR); variants of PCR such as Rapid amplification of cDNA ends (RACE), ligase chain reaction (LCR), multiplex RT-PCR, immuno-PCR, SSIPA, Real Time RT-qPCR and nanofluidic digital PCR; loop-mediated isothermal amplification (LAMP); recombinase polymerase amplification (RPA); isothermal amplification; Helicase-dependent isothermal DNA amplification (HDA); Rolling Circle Amplification (RCA); Nucleic acid sequence-based amplification (NASBA); strand displacement amplification (SDA); nicking enzyme amplification reaction (NEAR); polymerase Spiral Reaction (PSR); and others. In one example, devices can be used to detect a target nucleic acid for a diagnosis, e.g., for a SARS-CoV-2 diagnosis. In some implementations, a rotation or screw mechanism is contemplated for advancing a series of reactions. Some implementations allow for the preparation and addition of the third reagent at the point of use, and uses a brush-like mechanism to combine the smaller volumes and ensure their mixture. In some implementations, a positive displacement mechanism to add small volumes and beads to ensure effective mixing is used. In some implementations, seals (such as O-rings) can be used to prevent leakage. In some implementations, some reagents can be packaged at the factory and maintained in ‘blister pack’ compartments, e.g., under a foil seal that can be pierced during activation. In some implementations, the components can be designed to be injection molded in polyethylene or other plastics. Some implementations utilize a test strip, such as a lateral flow strip. In these implementations, the component housing the lateral flow strip is at least partially transparent, such that the lateral flow strip can be visually examined. In some implementations, an overall size of an exemplary device is approximately 20 cm in height.

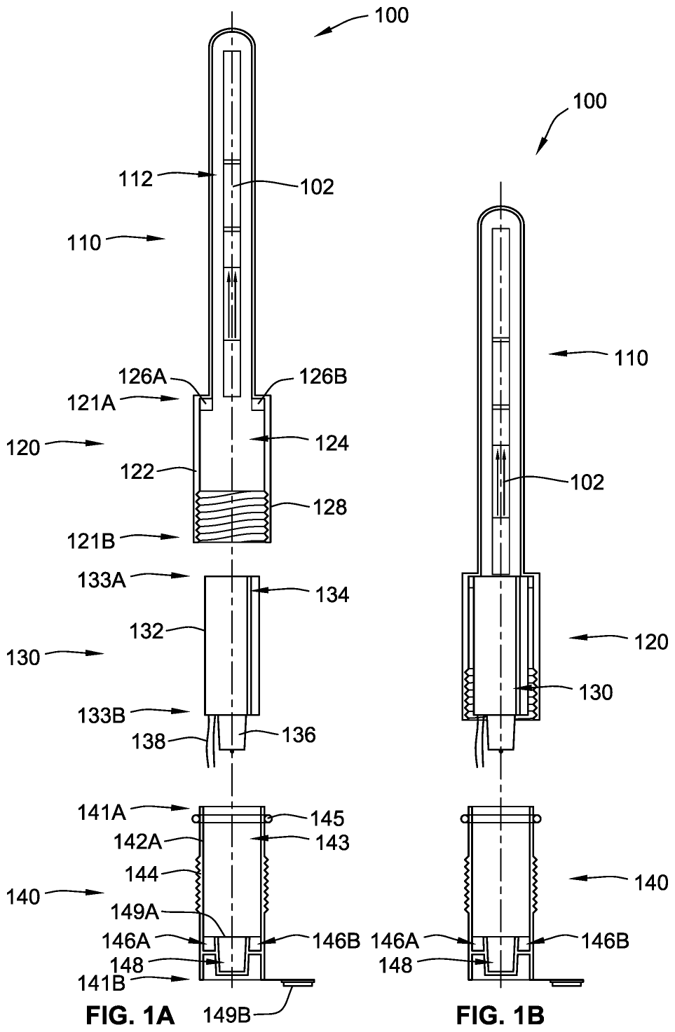

FIGS. 1A and 1B depict components of an exemplary device 100 for performing a multi-step assay. The device 100 includes a tube 110, a cap 120, an insert 130, and a reaction container 140. A lateral flow strip 102 (e.g., a test strip) is positioned within a hollow interior 112 of the tube 110. The lateral flow strip 102 can be any type of lateral flow strip used for a lateral flow immunoassay. In some implementations, the tube 110, the cap 120, the insert 130, and the reaction container 140 are injection molded in polyethylene or other plastics. In the illustrated implementations, the tube 110, the cap 120, the insert 130, and the reaction container 140 can have a circular cross-section.

In the illustrated implementation, the tube 110 and the cap 120 are unitary or monolithic. In this implementation, the tube 110 and the cap 120 are formed (for example via injection molding) as a single piece. In other implementations, the tube 110 and cap 120 are formed separately, and can then be coupled to each other. The cap 120 is formed from a cylindrical wall 122 that defines a hollow interior 124. The hollow interior 124 is generally open at both ends 121A, 121B of the cap 120, such that the hollow interior 124 is defined all the way through the cap 120. One end 121A of the cap 120 includes slots 126A and 126B, while the other end 121B of the cap 120 includes internal threads 128. As explained in more detail herein the slots 126A and 126B are configured to engage the insert 130, such that the insert 130 is rotationally locked to the cap 120 and cannot rotate relative to the cap 120.

The insert 130 is formed from a body 132 that includes one or more passageways or apertures 134 defined all the way through the body 132, from a first end 133A to a second end 133B. The body 132 of the insert 130 is configured to be received in the hollow interior 124 of the cap 120. The insert 130 further includes a displacing bump 136 and a brush 138. The displacing bump 136 and the brush 138 each extend away from the second end 133B of the body 132. The displacing bump 136 generally extends from the center of the second end 133B of the body 132. The displacing bump 136 is depicted as having a generally rectangular shape. However, the displacing bump 136 can have other shapes as well. For example, the displacing bump 136 can have a square shape, a cylindrical shape, a conical shape, a triangular shape, a trapezoidal shape, a frustum shape, etc.

In FIG. 1A, the brush 138 is depicted as being formed from bristles located only on one side of the of the second end 133B of the body 132. However, in some implementations, the brush 138 is formed from bristles located about the entire circumference of the second end 133B of the body 132. In these implementations, the bristles forming the brush 138 generally surround the displacing bump 136.

The reaction container 140 includes walls 142A and 142B that together define an internal cavity 143. In the illustrated implementation, the reaction container 140 has a circular cross-section. Thus, wall 142A can be a hollow cylindrical tube, while wall 142B is a circular base. An O-ring 145 can be located on the exterior circumference of the wall 142A, at a first end 141A of the reaction container 140. The reaction container 140 further includes external threads 144 located between the first end 141A and the second end 141B of the reaction container 140, so that the reaction container 140 can be coupled to the cap 120 via a threaded connection. The external threads 144 are configured to engage with the internal threads 128 of the cap 120, to thereby rotatably couple the reaction container 140 to the cap 120. The internal threads 128 and the external threads 144 could both be left-handed threads or both be right-handed threads. Moreover, in some implementations, the internal threads 128 and the external threads 144 could both be modified such that threads 128 are external threads and threads 144 are internal threads.

The reaction container 140 also contains a plurality of wells that includes wells 146A and 146B and central well 148. The wells 146A, 146B, and 148 are configured to store various substances therein, such as buffers and reagents. In one implementation, well 146A stores a recombinase polymerase amplification (RPA) reagent, well 146B stores a sodium dodecyl sulfate (SDS) reagent, and central well 148 stores an exonuclease reaction buffer.

Generally, the sample being tested is placed into the reaction container 140 prior to performing the assay, for example via the use of a pipette. In some implementations, the sample may be placed into one or both of the wells 146A and 146B. In other implementations, a portion of the sample is disposed in the hollow interior of the reaction container 140 above the wells 146A and 146B. In the illustrated implementations, wells 146A and 146B are separate wells that are not fluidly coupled together. However, in other implementations, the reaction container 140 may include a single toroidal well, instead of the two separate wells 146A and 146B.

In some implementations, the wells 146A and 146B are open at one end, with the other end being formed from the structure of the reaction container 140. In some implementations, the central well 148 is open at both ends (e.g., neither end of the central well 148 is formed from the structure of the reaction container 140. In these implementations, the reaction container 140 can further include a seal 149A covering one end of the central well 148, and a removable cap 149B covering the other end of the central well 148. In some implementations, seal 149A is a foil seal.

FIG. 1B depicts an implementation of the device 100 assembled for use by a user. The lateral flow strip 102 is positioned within the tube 110, while the insert 130 is positioned within the cap 120. The reaction container 140 remains separate from the insert 130. In some implementations, substances are already stored any one or more of the wells 146A and 146B, and the central well 148 when assembled as depicted in FIG. 1B. In other implementations, the reaction container does not have any substances stored therein when assembled as depicted in FIG. 1B.

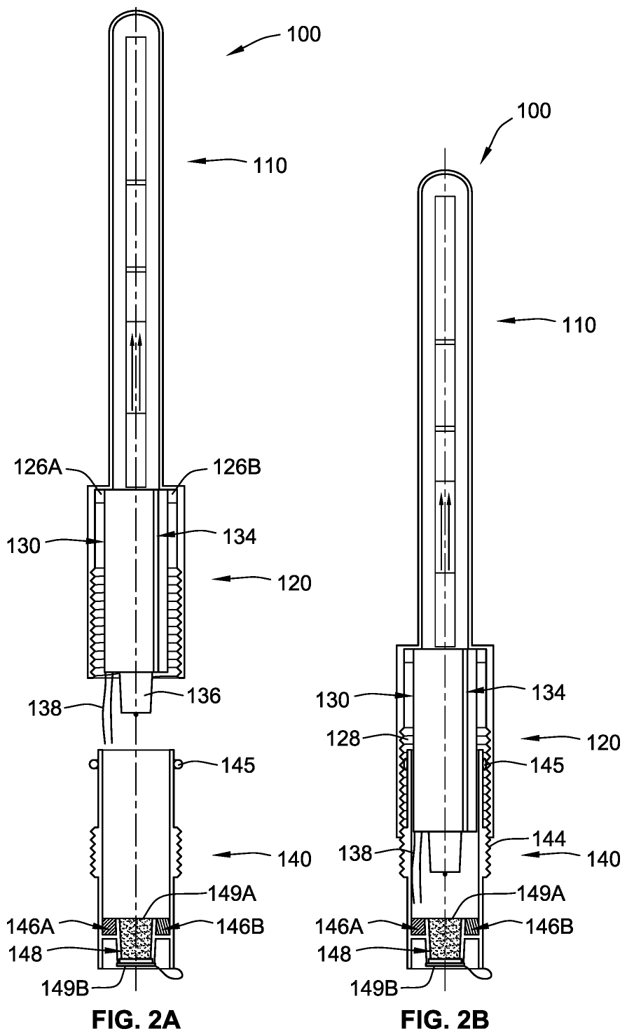

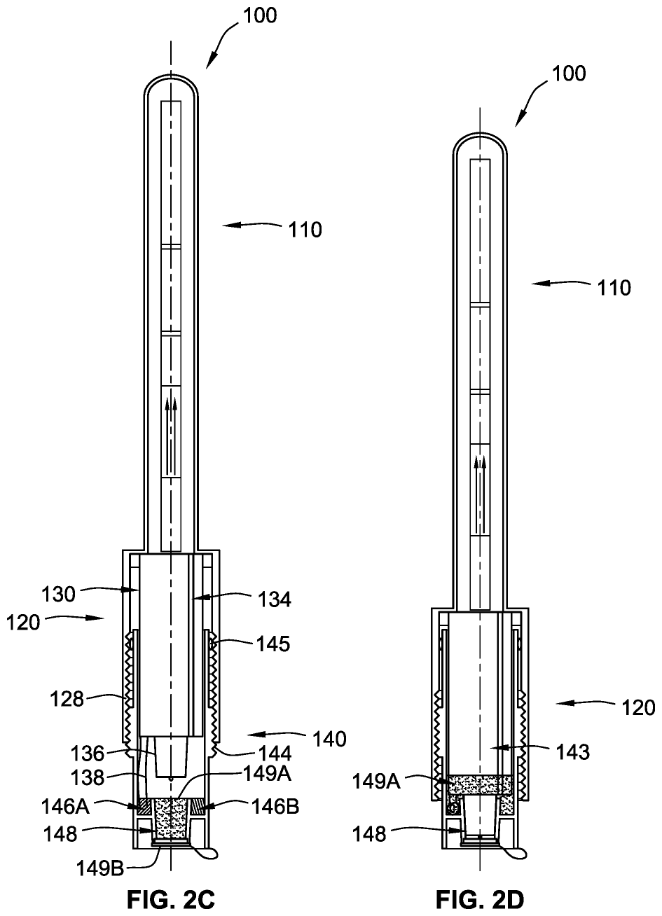

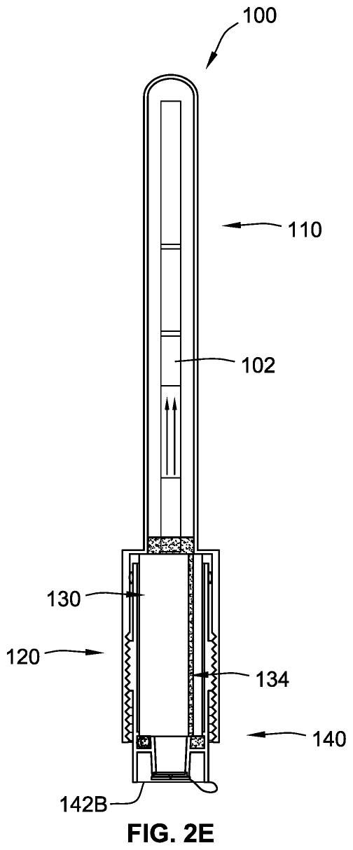

FIGS. 2A-2E depict the steps in some implementation for using the device 100 to perform a test, such as a multi-step assay. In FIG. 2A, one substance (such as RPA) is deposited into well 146A of the reaction container 140, and another substance (such as SDS) is deposited into well 146B of the reaction container 140. In the illustrated implementation, the central well 148 already contains a substance (such as the exonuclease reaction buffer), and is closed off on both ends by the seal 149A and the removable cap 149B. However, in other implementations, this step can include depositing the substance in the central well 148 and then sealing the central well 148. The sample being tested is also placed into the reaction container 140 (for example via a pipette) at this step, and the insert 130 is then inserted into the cap 120 such that the slots 126A and 126B engage the insert 130 and prevent the insert 130 from rotating relative to the cap 120. As shown, at least a portion of the displacing bump 136 and the brush 138 extend outward from the hollow interior 124 of the cap 120.

In FIG. 2B, the reaction container 140 has initially been coupled to the cap 120. In the illustrated implementation, the internal threads 128 of the cap 120 engage with the external threads 144 of the reaction container 140, so that the reaction container 140 can be screwed onto the cap 120. Here, the reaction container 140 is partially screwed onto the cap 120, so that the aperture 134 fluidly connects the hollow interior 124 of the cap 120, with the internal cavity 143 of the reaction container 140. The O-ring 145 is positioned within the hollow interior 124 of the cap 120. The displacing bump 136 and the brush 138 extend toward the wells 146A, 146B, and 148, but do not reach the wells 146A, 146B. Once the reaction container 140 is attached to the cap 120, the device 100 can be incubated. In one example, the device 100 is incubated at about 42° C. for about five minutes. Depending on the substances used and the desired test, the incubation temperature and/or time can be higher or lower.

In FIG. 2C, once the device 100 has been incubated, the reaction container 140 can be further screwed onto the cap 120 by rotating the reaction container 140 relative to the cap 120, to a first position relative to the cap 120, as shown in FIG. 2C. Due to the engagement of the internal threads 128 and the external threads 144, this rotation toward the first position causes the wall 142B to advance toward the second end 133B of the insert, thus decreasing the volume of the internal cavity 143 of the reaction container 140. In turn, the wells 146A and 146B advance toward the brush 138. As the reaction container 140 is rotated, the brush 138 contacts the substances in the wells 146A and 146A and aids in mixing together the substances (which in some implementations could include reagents) and the sample. As depicted in FIG. 2C, once the reaction container 140 is rotated to the first position relative to the cap 120, the brush 138 is in contact with the wells 146A and 146B, but the displacing bump 136 is still spaced apart from the central well 148.

In FIG. 2D, the reaction container 140 is rotated relative to the cap 120 to a second position relative to the cap 120. As this rotation occurs, the volume of the internal cavity 143 is further reduced, and the central well 148 advances toward displacing bump 136. When the displacing bump 136 reaches the central well 148, the displacing bump 136 breaks through the seal 149A of the central well 148. The substance stored in the central well 148 can then be mixed with the mixed substances from wells 146A and 146B and the sample. The rotation can aid in mixing the substance of the central well 148 with the mixed substances from wells 146A and 146B and the sample within the internal cavity 143, due at least in part to the rotation of the brush 138. The bristles that form the brush 138 are generally flexible, so that the bristles can bend out of the way. After this mixing, the device 100 can again be incubated. In one example, the device 10 is incubated for about one minute at room temperature (e.g., between about 20° C. and about 22° C.). Depending on the substances used and the desired test, the incubation temperature and/or time can be higher or lower.

In FIG. 2E, the reaction container 140 continues to be rotated from the second position toward a third position relative to the cap 120. The continued rotation toward the third position reduces the volume of the internal cavity 143. In turn, the reduced volume of the internal cavity 143 causes the mixture of the substances and the sample to travel through the aperture 134 in the insert 130, to the nearest end of the lateral flow strip 102 within the tube 110. Thus, rotation of the reaction container 140 relative to the cap 120 causes the substances within the reaction container 140 (e.g., the reagents and the buffer) and the sample to be mixed together, and further causes at least a portion of the mixed fluids to be delivered from the reaction container 140 to the lateral flow strip 102, via the insert 130.

Once the mixture reaches the lateral flow strip 102, the mixture begins to interact with the lateral flow strip 102 (e.g., one or more chemical reactions between the reagent/buffer mixture and the lateral flow strip 102 begins). The device 100 can again be incubated until the interaction is complete, at which time the lateral flow strip 102 can be examined to determine the results of the test. In some implementations, the tube 110 is transparent, so that the lateral flow strip 102 can be visually examined while disposed in the hollow interior 112 of the tube 110. Once the test is complete and the results are recorded, the entire device 100 can be discarded, or the device 100 can be sterilized and prepared for re-use.

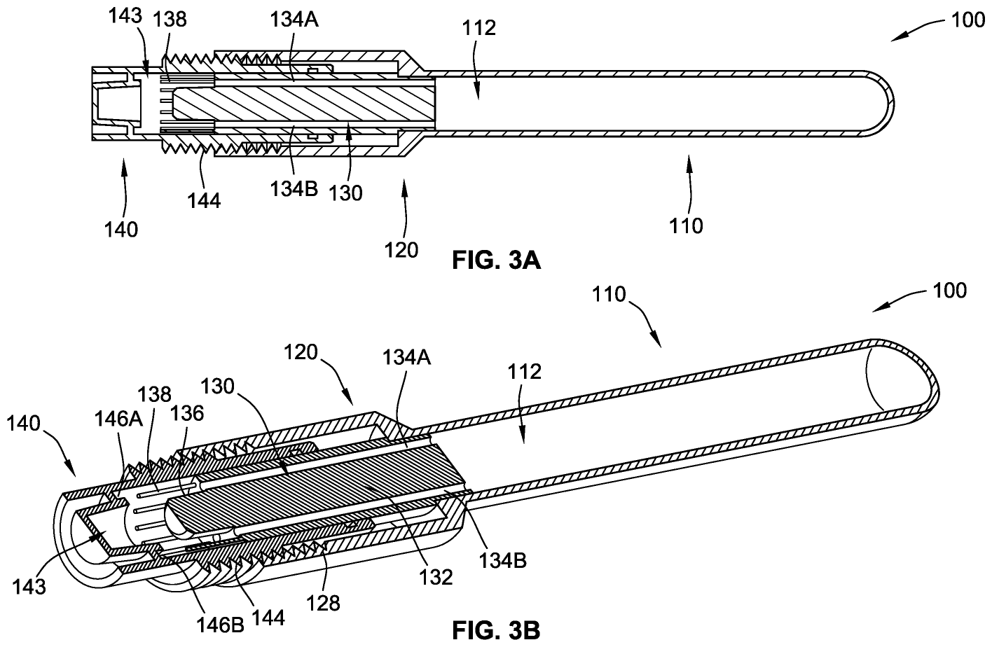

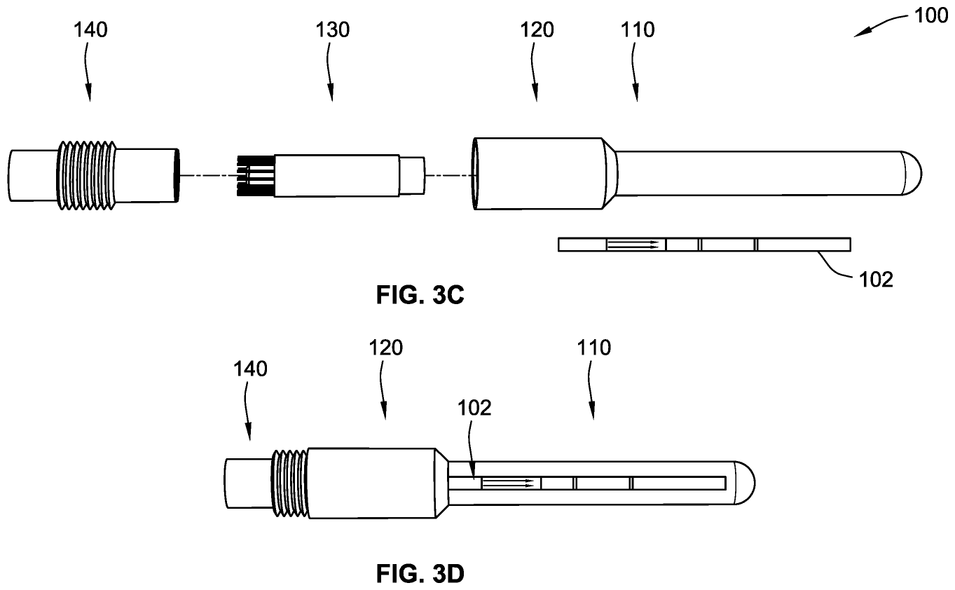

FIG. 3A depicts a side cross-sectional view of the exemplary device 100, and FIG. 3B depicts a perspective cross-sectional view of the exemplary device 100. In FIGS. 3A and 3B, the insert 130 is positioned within the hollow interior of the cap 120, and the reaction container 140 has been screwed onto the cap 120 via engagement of the internal threads 128 of the cap 120, and the external threads 144 of the reaction container 140. In FIGS. 3A and 3B, the reaction container 140 has been rotated almost to the first position, such that the brush 138 has almost reached the wells 146A and 146B. In the implementation illustrated in FIG. 3A, the insert 130 includes two apertures 134A and 134B, which are each similar to aperture 134 of FIGS. 1A and 1B. The apertures 134A and 134B extend between the ends of the body 132 of the insert 130. The apertures 134A and 134B thus fluidly connect the internal cavity 143 of the reaction container 140 with the hollow interior 112 of the tube 110.

FIG. 3C depicts an exploded perspective view of the device 100, showing the lateral flow strip 102, the tube 110, the cap 120, the insert 130, and the reaction container 140. In FIG. 3C, the cap 120 is a monolithic/unitary part of the tube 110. The brush 138 of the insert 130 is visible, along with the external threads 144 of the reaction container 140. FIG. 3D depicts a perspective view of the device 100 when reaction container 140 is attached to the cap 120, and the lateral flow strip 102 is positioned inside the tube 110.

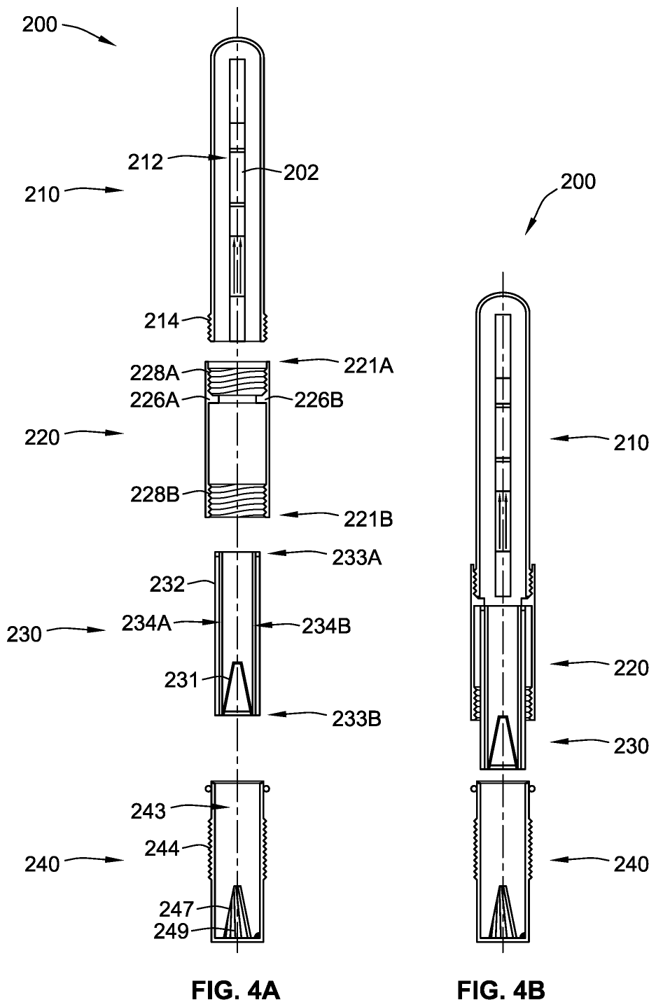

FIG. 4A depicts an exemplary device 200 for performing a multi-step assay. In some aspects, device 200 is generally similar to device 100, and includes a tube 210 with a hollow interior 212 that contains a lateral flow strip 202 (e.g., a test strip), a cap 220, an insert 230, and a reaction container 240. In device 200, the cap 220 is specifically a separate piece from the tube 210. The tube 210, the cap 220, and the reaction container 240 all contains threads, so that the tube 210 and the cap 220 can be coupled via a threaded connection, and so that the cap 220 and the reaction container 240 can be coupled via a threaded connection. The tube 210 contains external threads 214 that are configured to engage with a first set of internal threads 228A located at a first end 221A of the cap 220, to thereby rotatably couple the cap 220 to the tube 210. The cap 220 also contains a second set of internal threads 228B located at a second end 221B of the cap 220 that are configured to engage with external threads 244 of the reaction container 240, to thereby rotatably couple the reaction container 240 to the cap 220. The cap 220 includes slots 226A and 226B configured to engage the insert 230, and prevent the insert 230 from rotating relative to the cap 220. Generally, any pair of threads of device 200 that engage with each other can both be left-handed, or can both be right-handed. In some implementations, the upper pair of threads (formed by external threads 214 and the first set of internal threads 228A) has the same handedness as the lower pair of threads (formed by the second set of internal threads 228AB and the external threads 244). In other implementations, the upper pair of threads has the opposite handedness as the lower pair of threads. Moreover, while the threads 214, 228A, 228B, and 244 are shown illustrated as being internal or external, the orientation of the threads could be modified as desired. In one example, threads 214 could be internal threads and threads 228A could be external threads. In another example, threads 228B could be external threads and threads 244 could be internal threads.

In the implementation illustrated in FIG. 4A, the insert 230 is formed from a body 232 having a blister pack 231 located at end 233B of the body 232. End 233B of the body 232 is closest to the reaction container 240, while end 233A of the body 232 is closest to the cap 220. The insert 230 also includes two apertures 234A and 234B defined through the body 232. The blister pack 231 contains a substance (such as an exonuclease reaction buffer). The reaction container 240 can store a substance (such as RPA) within the internal cavity 243 of the reaction container 240. The sample being tested can also be placed into the internal cavity 243 of the reaction container 240 prior to performing the assay, for example via a pipette. The reaction container 240 can further include a protrusion 247 that is configured to engage (e.g., pierce) the blister pack 231 of the insert 230 and release the substance. In the illustrated implementation, the blister pack 231 and the protrusion 247 are generally conical, although they can have other shapes as well. The protrusion 247 can include one or more vanes 249 extending from a surface of the protrusion 247. The vanes 249 aid in mixing the substances once the protrusion 247 pierces the blister pack 231. The vanes 249 can be arranged in a helical pattern, a semi-helical pattern, a vertical pattern, a horizontal pattern, a diagonal pattern, and other patterns. The vanes 249 can further be arranged in any combination of these different patterns.

FIG. 4B depicts an implementation of the device 200 as assembled for use by a user. The cap 220 is screwed into the tube 210, and the insert 230 is inserted into the hollow interior 212 of the cap 220. The reaction container 240 can remain separate.

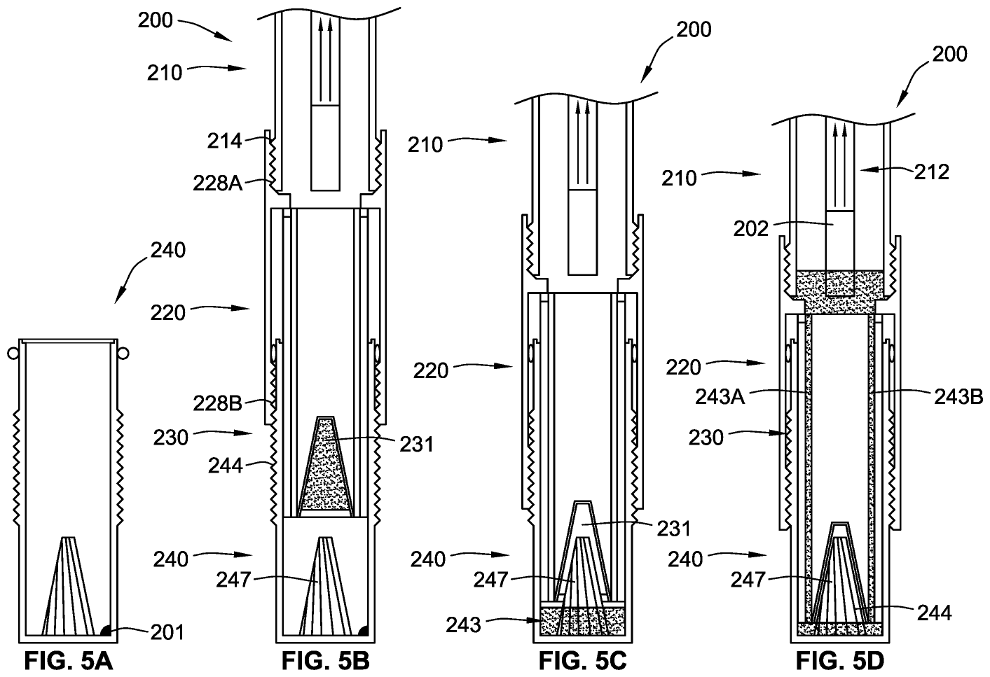

FIGS. 5A-5D depict the steps for using the device 200 to perform a test, such as a multi-step assay. In FIG. 5A, a substance (shown as 201) can be deposited into the reaction container 240. The substance 201 could be a reagent such as RPA. Generally, the sample being tested is also be placed into the reaction container 240 at this step, for example using a pipette. In FIG. 5B, the cap 220 has been screwed onto the tube 210 (via the external threads 214 and the first set of internal threads 228A), and the reaction container 240 has been screwed onto the cap 220 (via the second set of internal threads 228B and the external threads 244). Here, the reaction container 240 is positioned so that the protrusion 247 does not engage the blister pack 231 of the insert 230. At this step depicted in FIG. 5B, the device 200 can be incubated, for example at about 42° C. for about five minutes.

In FIG. 5C, the reaction container 240 is further rotated relative to the cap 220 to a first position relative to the cap 220. Rotation to the first position advances the protrusion 247 toward the blister pack 231, so that the protrusion 247 engages (e.g. pierces) the blister pack 231, and releases the substance (such as the exonuclease reaction buffer) within the blister pack 231 into the internal cavity 243 of the reaction container 240.

In FIG. 5D, the reaction container 240 is further rotated relative to the cap 220 to a second position relative to the cap 220. The vanes 249 extending from the surface of the protrusion 247 aid in mixing the substances and the sample, as the reaction container 240 is rotated to the second position. The rotation also forces the mixture of the substances and the sample through the apertures 234A and 234B in the body 232 of the insert 230 and into the hollow interior 212 of the tube 210, as the rotation reduces the volume of the internal cavity 243 of the reaction container 240. When the mixture is forced into the hollow interior 212 of the tube 210, the mixture contacts the lateral flow strip 202 and begin to interact with the lateral flow strip 202. Once the interaction is complete, the lateral flow strip 202 can be examined to determine the results of the test. In some implementations, the tube 210 is transparent, so that the lateral flow strip 202 can be visually examined while disposed in the hollow interior 212 of the tube 210. Once the test is complete and the results are recorded, the entire device 200 can be discarded, or the device 200 can be sterilized and prepared for re-use.

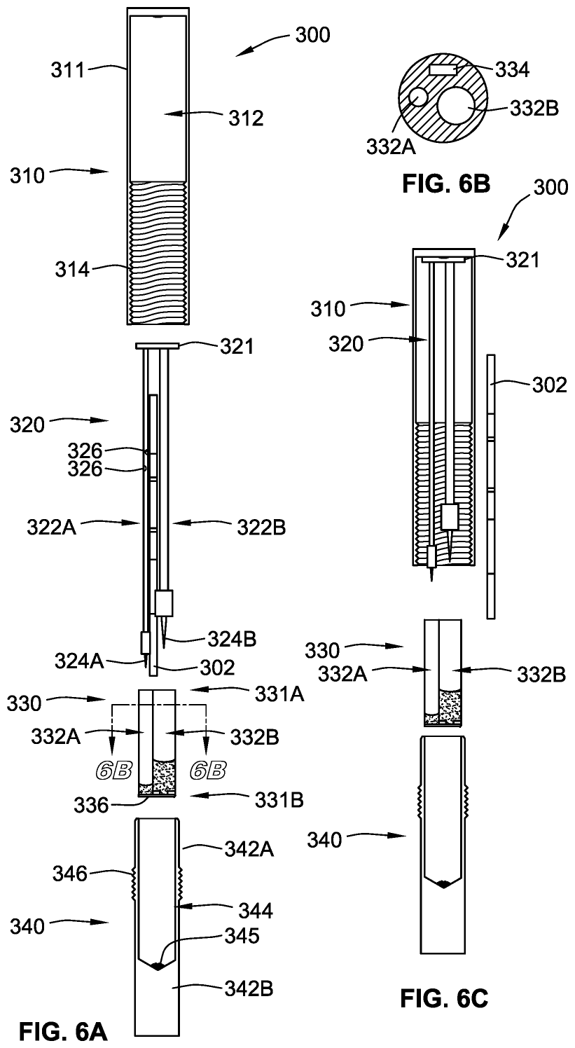

FIG. 6A depicts an exemplary device 300 for performing a multi-step assay. In some aspects, the device 300 can be similar to device 100 and device 200. The device 300 includes a cap 310, a plunger assembly 320, a reagent insert 330, and a reaction container 340. The cap 310 is formed from a cylindrical wall 311 that defines a hollow interior 312. The cap 310 also includes internal threads 314. The hollow interior 312 is generally open at least one end of the cap 310, such that the hollow interior 312 is defined at least partially through the cap 310. At least a portion of a lateral flow strip 302 (e.g., a test strip) can be positioned within the hollow interior 312 of the cap 310 during use.

The plunger assembly 320 includes a primary plunger 322A and a secondary plunger 322B coupled to a base 321. The plunger assembly 320 is configured to be received within the hollow interior 312 of the cap 310. The primary plunger 322A is longer than the secondary plunger 322B, such that a tip 324A of the primary plunger 322A is spaced farther apart from the base 321 than a tip 324B of the secondary plunger 322B. As discussed in more detail herein, the primary plunger 322A is configured to buckle or compress in response to a sufficient amount of force being applied to the plunger, directed from the tip 324A toward the base 321. Thus, in some implementations, the primary plunger 322A has one or more buckle points. In the illustrated implementation, the buckle points are depicted as notches 326 that are cut out of the primary plunger 322A. When sufficient force is applied to the primary plunger 322A, the primary plunger 322A can bend or crease at these notches 326, such that the primary plunger 322A buckles and can be compressed.

While the illustrated implementation depicts notches 326 cut out of the primary plunger 322A, other types of buckle points can be used. For example, material at portions of the primary plunger 322A could be fabricated to be weaker (such as by adding perforation) instead of being cut out, to cause the primary plunger 322A to buckle at those points. In another example, at least a portion of the primary plunger 322A has a spring-like structure, such that that portion of the primary plunger 322A is configured to compress when the tip 324A of the primary plunger 322A reaches the lower end of the internal cavity 344 (e.g., the upper end of the base 342B).

The reagent insert 330 includes a primary aperture 332A and a secondary aperture 332B defined therethrough. The primary aperture 332A and the secondary aperture 332B generally extend the entire length of the reagent insert 330, from a first end 331A to a second end 331B.

FIG. 6B depicts a top view of the first end 331A of the reagent insert 330. As shown, the first end 331A includes openings for the primary aperture 332A and the secondary aperture 332B. However, the first end 331A also includes an opening for a slot 334 that is defined from the first end 331A to the second end 331B. The second end 331B also has three openings, for the primary aperture 332A, the secondary aperture 332B, and the slot 334. The slot 334 is configured to receive at least a portion of the lateral flow strip 302.

Referring back to FIG. 6A, the reagent insert 330 further includes a seal 336 disposed at the second end 331B. The seal 336 (which could be a foil seal) covers the openings of the primary aperture 332A and the secondary aperture 332B. With the seal 336 covering the openings in the second end 331B, the primary aperture 332A and the secondary aperture 332B are each configured to hold a substance (such as a reagent, a buffer, etc.). The primary aperture 332A is configured to receive the primary plunger 322A, while the secondary aperture 332B is configured to receive the secondary plunger 322B. In the illustrated implementation, the primary plunger 322A has a first plunger diameter and the primary aperture 332A has a first aperture diameter. The first plunger diameter of the primary plunger 322A is less than or equal to the first aperture diameter of the primary aperture 332A. The secondary plunger 322B has a second plunger diameter and the secondary aperture 332B has a second aperture diameter. The second plunger diameter of the secondary plunger 322B is less than or equal to the second aperture diameter of the secondary aperture 332B. The second plunger diameter of the secondary plunger 322B is larger than the first plunger diameter of the primary plunger 322A and the first aperture diameter of the primary aperture 332A. Thus, the secondary plunger 322B cannot inadvertently be inserted into the primary aperture 332A during use.

The reaction container 340 is generally formed from a cylindrical wall 342A and a base 342B, that define an internal cavity 344. The upper end of the base 342B (e.g., nearer to the external threads 346) forms the lower end of the internal cavity 344 (e.g., further away from the external threads 346). The internal cavity 344 is configured to hold various substances. In one example, the internal cavity 344 may hold RPA and one or more small beads 345, which can aid in mixing the RPA with other substances during use of the device 300. The sample being tested can also be placed into the reaction container 240 prior to performing the assay, for example via a pipette. The reaction container 340 also contains external threads 346, so that the reaction container 340 can be coupled to the cap 310 via a threaded connection. The external threads 346 of the reaction container 340 are configured to engage with the internal threads 314 of the cap 310, to thereby rotatably couple the reaction container 340 to the cap 310. In some implementations, the reaction container 340 includes an external O-ring configured to form a seal between the exterior of the reaction container 340 and the interior of the cap 310, when the reaction container 340 and the O-ring are positioned within the hollow interior 312 of the cap 310. The internal threads 314 and the external threads 346 could both be left-handed threads or both be right-handed threads. Moreover, in some implementations, the internal threads 314 and the external threads 346 could both be modified such that threads 314 are external threads and threads 346 are internal threads.

FIG. 6C depicts an implementation of the device 300 as assembled for use by a user. As shown, the plunger assembly 320 is positioned at least partially within the hollow interior 312 of the cap 310. In some implementations, the cap 310 and the base 321 of the plunger assembly may having features that allow the plunger assembly 320 to be coupled to the cap 310. The reagent insert 330, the reaction container 340, and the lateral flow strip 302 can all remain separate when assembled as depicted in FIG. 6C.

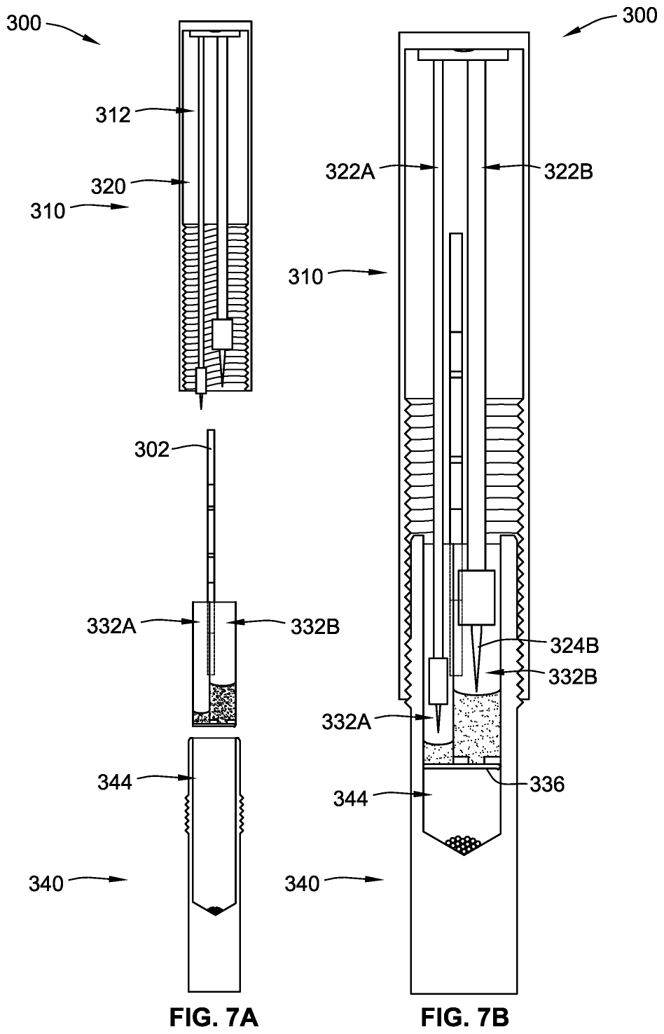

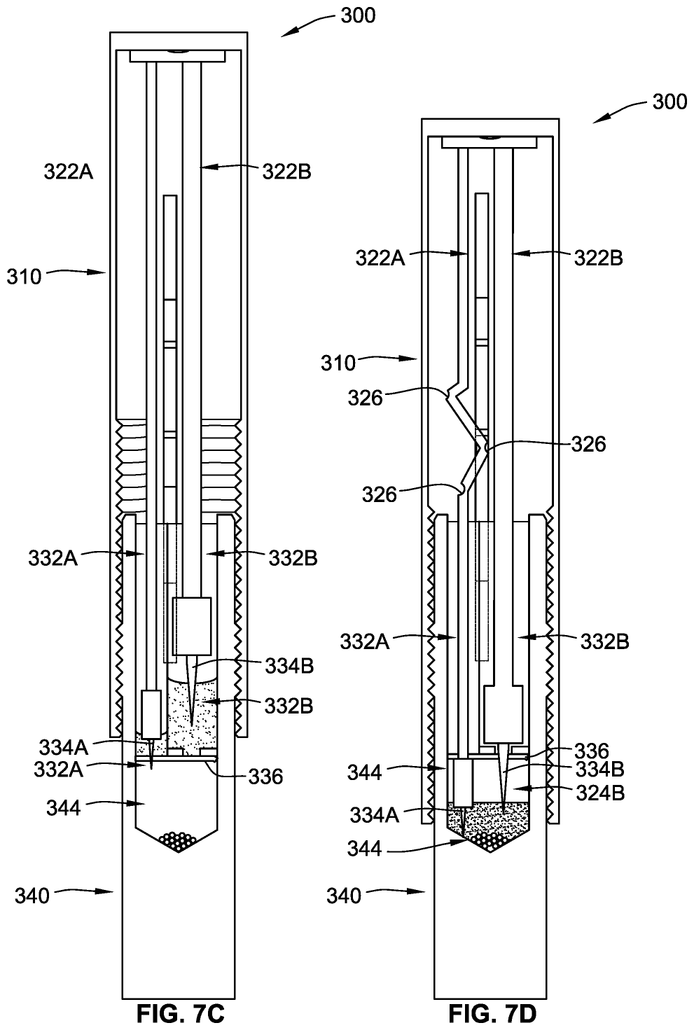

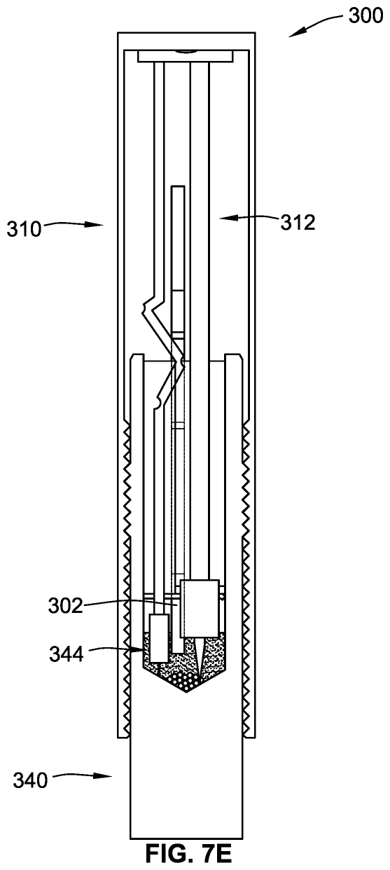

FIGS. 7A-7E depict the steps for using the device 300 to perform a test, such as a multi-step assay. In FIG. 7A, a substance (such as RPA) has been deposited into the internal cavity 344 of the reaction container 340, a substance (such as SDS) has been deposited into the primary aperture 332A of the reagent insert 330, and a substance (such as the exonuclease reaction buffer) has been deposited into the secondary aperture 332B of the reagent insert 330. Generally, the sample being tested will also be placed into the reaction container 340 at this step, for example using a pipette. The lateral flow strip 302 has also been inserted into the slot of the reagent insert 330. The plunger assembly 320 is positioned within the hollow interior 312 of the cap 310. In FIG. 7B, the reaction container 340 has been initially screwed onto the cap 310. The primary plunger 322A has been inserted into the primary aperture 332A, while the secondary plunger 322B has been inserted into the secondary aperture 332B. However, neither the tip 324A of the primary plunger 322A, nor the tip 324B of the secondary plunger 322B has reached the seal 336. The device 300 can then be incubated, for example at about 42° C. for about 5 minutes.

In FIG. 7C, the reaction container 340 has been rotated relative to the cap 310, such that the reaction container 340 is in a first position relative to the cap 310. Rotating to the first position causes the plunger assembly 320 and the reagent insert 330 to move toward each other, such that the plunger assembly 320 is closer to the seal 336. Because the primary plunger 322A is longer than the secondary plunger 322B, the tip 324A of the primary plunger 322A reaches the seal 336 before the tip 324B of the secondary plunger 322B. The tip 324B pierces the portion of the seal 336 that covers the primary aperture 332A, which allows the substance in the primary aperture 332A to move into the internal cavity 344 of the reaction container 340. Because the tip 324B of the secondary plunger 322B does not reach the seal 336, the portion of the seal 336 covering the secondary aperture 332B remains intact. The substance from the primary aperture 332A, the reaction container 340, and the sample can be mixed, for example by gently shaking the device 300. In some implementations, the primary plunger 322A aids in mixing the two substances and the sample, as the tip 324A of the primary plunger 322A advances past the seal 336. The device 300 can then be incubated, for example at room temperature (e.g., between about 20° C. and about 22° C.).

In FIG. 7D, the reaction container 340 has been rotated relative to the cap 310, such that the reaction container 340 is in a second position relative to the cap 310. Rotating to the second position causes the plunger assembly 320 and the reagent insert 330 toward each other, such that the primary and secondary plungers 322A and 322B are closer to the reagent insert 330. The primary plunger 322A advances until it contacts the lower end of the internal cavity 344 (e.g., the upper end of the base 342B). Because of the notches 326 cut out of the primary plunger 322A, the primary plunger 322A buckles, and thus does not prevent the secondary plunger 322B from further advancing toward the seal 336. As the secondary plunger 322B reaches the seal 336, the tip 324B of the secondary plunger 322B pierces the portion of the seal 336 covering the secondary aperture 332B. The substance stored in the secondary aperture 332B is then allowed to move into the internal cavity 344 of the reaction container 340, along with the already-mixed combination of the sample and the substances from the internal cavity 344 and the primary aperture 332A. The sample and all of the substances can be further mixed, for example by gently shaking the device 300. In some implementations, the primary plunger 322A and the secondary plunger 322B aid in mixing the two substances, as the tip 324A of the primary plunger 322A remains advanced past the seal 336, and as the tip 324B of the secondary plunger 322B advances past the seal 336. The device 300 can then incubated, for example, at room temperature (e.g., between about 20° C. and about 22° C.).

In FIG. 7E, the reaction container 340 has been rotated relative to the cap 310, such that the reaction container 340 is in a third position relative to the cap 310. Rotating to the third position causes the mixture of the sample and the other substances in the internal cavity 344 of the reaction container 340 to contact the lateral flow strip 302 within the cap 310. The mixture contacts the lateral flow strip 302 and begin to interact with the lateral flow strip 302. Once the interaction is complete, the lateral flow strip 302 can be examined to determine the results of the test. In some implementations, the cap 310 is transparent, so that the lateral flow strip 302 can be visually examined while disposed in the hollow interior 312 of the cap 310. Once the test is complete and the results are recorded, the entire device 300 can be discarded, or the device 300 can be sterilized and prepared for re-use.

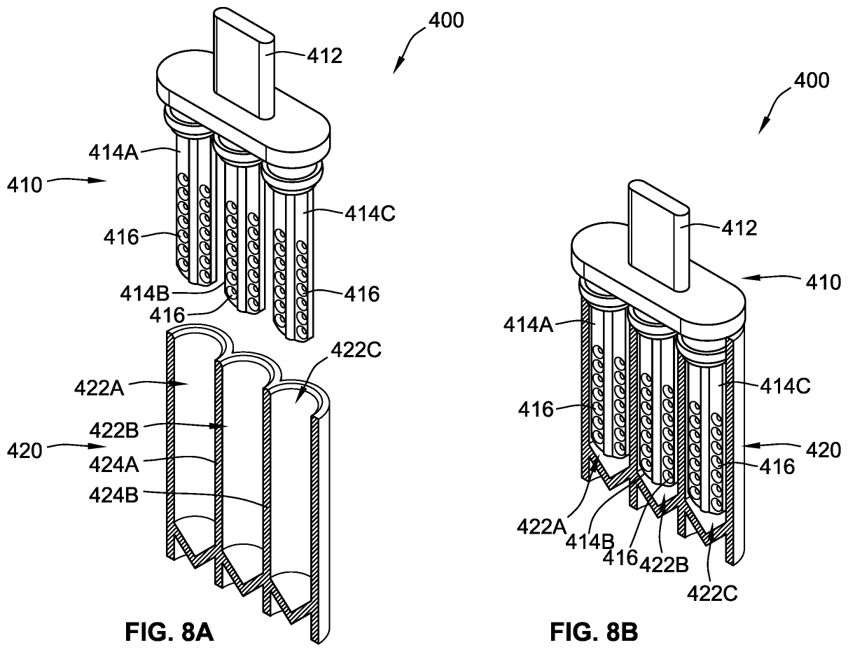

FIGS. 8A and 8B depict an exemplary device 400 for simultaneously performing a plurality of different tests on a sample. The device 400 includes a collection assembly 410 and a reaction container 420. The reaction container 420 is depicted as a cross-section in FIGS. 8A and 8B. The collection assembly 410 includes a handle 412 and three separate collection swabs 414A, 414B, and 414C that extend from the handle 412. Each of the collection swabs 414A-414C has a generally rectangular profile in the illustrated implementation, but could have a profile having one or more different shapes in other implementations. The collection swabs 414A-414C are arranged linearly, such that collection swab 414B is positioned between collection swab 414A and collection swab 414C along a single linear axis. Each of the collection swabs 414A-414C includes two parallel rows of apertures 416 defined therein. The apertures 416 are able to hold drops of liquid, which allows the collection swabs 414A-414C to more easily collect samples from a sample source. The collection swabs 414A-414C thus act as inoculation loops to collect samples.

The reaction container 420 includes three separate reaction chambers 422A, 422B, and 422C. The reaction chambers 422A-422C are arranged linearly, similarly to the collection swabs 414A-414C, such that reaction chamber 422B is positioned between reaction chamber 422A and reaction chamber 422C along a single linear axis. The reaction chambers 422A and 422B are separated from each other by a wall 424A. The reaction chambers 422B and 422C are separated from each other by a wall 424B. While the reaction container 420 is depicted as a cross-section to show the interior of the reaction chambers 422A-422C, each of the reaction chambers 422A-422C is enclosed on the bottom and the sides, and has a generally cylindrical profile. Each of the reaction chambers 422A-422C has a diameter that is greater than or equal to the width of the rectangular profile of the collection swabs 414A-414C, to allow the collection swabs to be inserted into the reaction chambers 422A-422C. However, the reaction chambers 422A-422C could have a profile having one or more different shapes in other implementations. The device 400 can be formed from any suitable material, such as plastic.

FIG. 8A shows the device 400 in an unassembled configuration (for example, prior to any tests being performed). As is shown, each of the collection swabs can be aligned over a respective one of the reaction chambers. Collection swab 414A is aligned over reaction chamber 422A. Collection swab 414B is aligned over reaction chamber 422B. Collection swab 414C is aligned over reaction chamber 422C. FIG. 8B shows the device 400 when the configuration of the device 400 has moved to an assembled configuration (for example, during or after tests have been performed). The collection assembly 410 is coupled to the reaction container 420 such that each of the collection swabs is 414A-414C inserted into one of the reaction chambers 422A-422C of the reaction container 420. The collection swab 414A is disposed in the reaction chamber 422A. The collection swab 414B is disposed in the reaction chamber 422B. The collection swab 414C is disposed in the reaction chamber 422C. The handle 412 covers the upper openings of the reaction chambers 422A-422C, such that the collection assembly 410 acts as a cap for the reaction container 420. While device 400 is shown with three collection swabs 414A-414C and three reaction chambers 422A-422C, the device 400 may include any suitable number of collection swabs and reaction chamber. In the illustrated implementation, each reaction chamber 422A-422C is associated with a corresponding one of the collection swabs 414A-414C, and receives that corresponding one the collection swabs 414A-414C when the collection assembly 410 is coupled to the reaction container in the assembled configuration. Thus, as shown in FIG. 8B, when the device 400 is moved to the assembled configuration, each of the reaction chambers 422A-422C at least partially houses the corresponding one of the plurality of collection swabs 414A-414C therein. However, in some implementations, at least one reaction chamber can be configured to receive multiple collection swabs, and/or at least one collection swabs can be configured to be received by multiple reaction chambers.

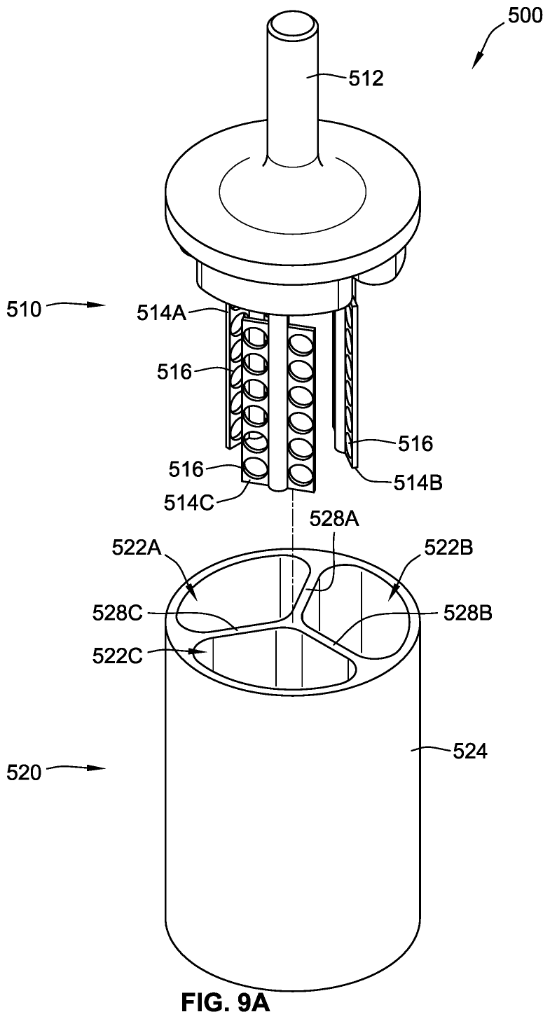

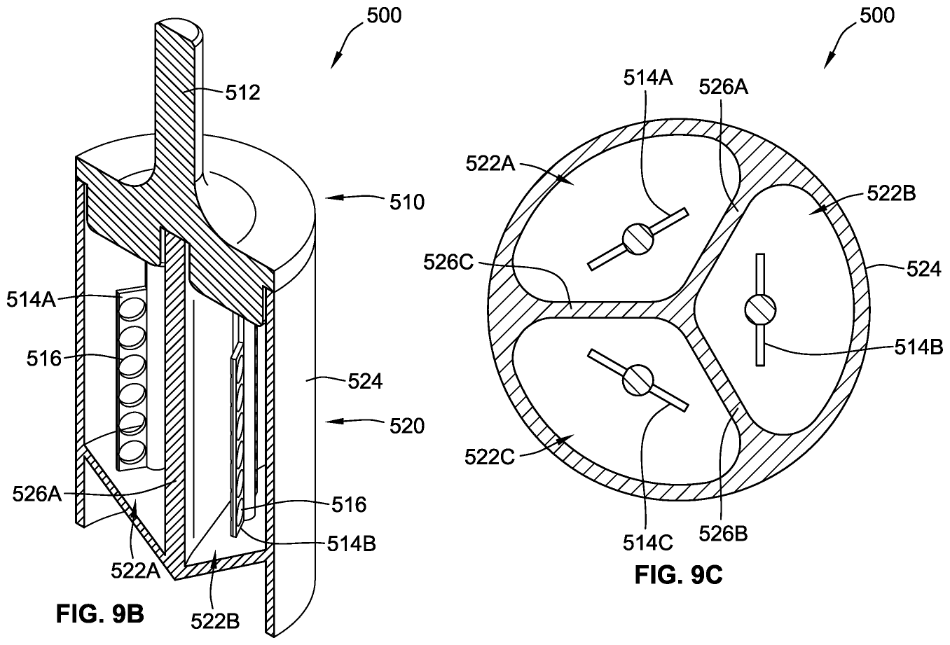

FIGS. 9A-9C depict an exemplary device 500. Similar to device 400, device 500 includes a collection assembly 510 and a reaction container 520. The collection assembly 510 includes a handle 512 and three separate collection swabs 514A, 514B, and 514C extending from the handle 512. Each of the collection swabs 514A-514C has a generally rectangular profile in the illustrated implementation, but could have a profile having one or more different shapes in other implementations. The collection swabs 514A-514C are arranged circularly, and are generally spaced evenly about the circumference of the circular shape defined by the outer bounds of the collection swabs 514A-514C. However, the collection swabs 514A-514C could be spaced differently about the circumference of this circular shape in other implementations. The collection swabs 514A-514C each include two parallel rows of apertures 516, similar to device 400.

The reaction container 520 has a cylindrical profile, and includes three separate reactions chambers 522A, 522B, and 522C. Similar to the collection swabs 514A-514C, the reaction chambers 522A-522C are arranged circularly, and are generally spaced evenly about the circumference of the cylindrical shape of the reaction container 520. However, the reaction chambers 522A-522C could be spaced differently about the circumference of the cylindrical shape of the reaction container 520 in other implementations. Each of the reaction chambers 522A-522C has a profile that is generally triangular (or pie-shaped) with rounded corners. The smallest dimension of the triangular (or pie-shaped) profile of the reaction chambers 522A-522C is greater than or equal to the width of the rectangular profile of the collection swabs 414A-414C, to allow the collection swabs 514A-514C to be inserted into the reaction chambers 522A-522C. However, the reaction chambers 522A-522C could have a profile having one or more different shapes in other implementations. The device 400 can be formed from any suitable material, such as plastic.

The reaction container 520 is formed by an outer cylindrical wall 524, and three inner walls 526A, 526B, and 526C. Reaction chamber 522A is defined by outer wall 525, inner wall 526A, and inner wall 526C. Reaction chamber 522B is defined by outer wall 525, inner wall 526A, and inner wall 526B. Reaction chamber 522C is defined by outer wall 525, inner wall 526B, and inner wall 526C. Inner wall 526A forms a barrier between reaction chambers 522A and 522B. Inner wall 526B forms a barrier between reaction chambers 522B and 522C. Inner wall 526C forms a barrier between reaction chambers 522A and 522C. Similar to device 400, the device 500 can be formed from any suitable material, such as plastic.

FIG. 9A shows the device 500 in an unassembled configuration (for example, prior to any tests being performed). As is shown, each of the collection swabs can be aligned over a respective one of the reaction chambers. Collection swab 514A is aligned over reaction chamber 522A. Collection swab 514B is aligned over reaction chamber 522B. Collection swab 514C is aligned over reaction chamber 522C. FIG. 9B and FIG. 9C show the device 500 when the configuration of the device 500 has moved to an assembled configuration (for example, during or after tests have been performed). FIG. 9B is a cut-away view that shows the interior of the reaction container 520. FIG. 9C is a top cross-sectional view that shows the collection swabs 514A-514C and the reaction chambers 522A-522C. When assembled, the collection assembly 510 is coupled to the reaction container 520 such that each of the collection swabs 514A-514C is inserted into one of the reaction chambers 522A-522C of the reaction container 520.

As can be seen, particularly in FIG. 9C, the reaction container 520 has a generally circular cross-section, and each of the reaction chambers 522A-522C occupies a portion of the reaction chamber 520 that spans about 120° of the circumference of the reaction chamber 520. The collection swabs 514A-514C are arranged in a corresponding fashion, such that each of the collection swabs 514A-514C is disposed within the 120° span of a respective one of the reaction chambers 522A-522C. Due to the presence of the walls 526A-526C, the actual span of the reaction chambers 522A-522C will generally be slightly less than 120°, depending on the thickness of the walls 526A-526C. Thus, each of the reaction chambers 522A-522C will generally occupy a portion of the reaction container 520 that spans between about 100° and about 120° of the circumference of the reaction container 520.

The collection swab 514A is disposed in the reaction chamber 522A. The collection swab 514B is disposed in the reaction chamber 522B. The collection swab 514C is disposed in the reaction chamber 522C. The handle 512 covers the upper openings of the reaction chambers 522A-522C, such that the collection assembly 510 acts as a cap for the reaction container 520. While device 500 is shown with three collection swabs 514A-514C and three reaction chambers 522A-522C, the device 500 may include any suitable number of collection swabs and reaction chambers. In the illustrated implementation, each reaction chamber 522A-522C is associated with a corresponding one of the collection swabs 514A-514C, and receives that corresponding one of the collection swabs 514A-514C when the collection assembly 510 is coupled to the reaction container 520 in the assembled configuration. Thus, as shown in FIG. 9B, when the device 500 is moved to the assembled configuration, each of the reaction chambers 522A-522C at least partially houses the corresponding one of the plurality of collection swabs 514A-514C therein. However, in some implementations, at least one reaction chamber can be configured to house multiple collection swabs in the assembled configuration, and/or at least one collection swab can be configured to be housed by multiple reaction chambers in the assembled configuration.

Devices 400 and 500 can be used to perform a variety of different tests on a variety of different samples. In some implementations, the collection swabs 414A-414C and 514A-514C can be used as oral collection swabs, and are configured to collect samples from a human mouth. In other implementations, the collection swabs 414A-414C and 514A-514C can be used as nasal collection swabs, and are configured to collect samples from a human nasal cavity. In additional implementations, the collection swabs 414A-414C and 514A-514C can be used as nasopharyngeal collection swabs, and are configured to collect samples from a human nasopharynx. In further implementations, the collection swabs 414A-414C and 514A-514C can be used as non-human collection swabs, and can be used to collect samples from other sources (such as bacteria samples growing on media plates or from liquid media).

Each of the reaction chambers 422A-422C and/or 522-522C can include any substance (or substances) that may be required to perform a desired test using device 500 or device 500. In some implementations, the reaction chambers of device 400 and/or device 500 are configured to perform the same assay with the same primer. In other implementations, the reaction chambers of device 400 and/or device 500 are configured to perform the same assay but with different primers. In further implementations, the reaction chambers of device 400 and/or device 500 are configured to perform different assays. In additional implementations, the reaction chambers of device 400 and/or device 500 are configured to perform any combination of assays. In some implementations, the substance or substances in the reaction chambers 422A-422C and/or 522A-522C are stored in a blister pack that is configured to be pierced by one of the collection swabs 414A-414C and/or 514A-514C when the collection assembly 410 and/or 510 is inserted into the reaction container 420 and/or 520. The substance or substances in the reaction chambers 422A-422C and/or 522A-522C can be wet, dry (e.g., lyophilized), or a combination of both.

In some implementations, devices 400 and 500 can include a mixing mechanism to allow for homogeneous reaction volumes. If the sample on the collection swab is not mixed evenly with the substance in the reaction chamber, the end test result may not be accurate. In some implementations, the mixing mechanism includes one or more beads that may be made from glass or metal. The beads can be pre-packaged in the reaction chambers. The beads can be configured to mix the sample and the substance in the reaction chamber with or without manual movement of the devices 400 and 500 (e.g., the user shaking or rotating the device). In other implementations, the mixing mechanism includes paddles within the reaction chambers. In some of these other implementations, the paddles are formed on or by the collection swabs. The paddles can be configured to move automatically to mix the sample and the substance, or can be configured to move in response to user action. In further implementations, the devices 400 and 500 can be configured such that user action causes mixing of the sample and the substance in response to manual movement of the devices 400 and 500. For example, the devices 400 and 500 can include one or more openings between separate reaction chambers, such that manual movement by the user causes the sample and/or the substance to flow between the chambers. Thus, devices 400 and 500 can include a plurality of mixing mechanisms that are each configured to aid in mixing (i) any substance in a corresponding one of the reaction chambers with (ii) the sample contained by the corresponding collection swab that is associated with the corresponding one of the reaction chambers.

In some implementations, the reaction chambers include a filter membrane and/or a bead column that act as a sample lysis mechanism. For example, if the test to be performed is a nucleic acid amplification reaction, the sample lysis mechanism allows the sample to undergo an RNA extraction process before the nucleic acid amplification reaction begins. Sample flow through the lysis mechanism can be driven by gravity, molecular forces, air pressured generated by coupling the collection assembly to the reaction container, or any combination thereof.

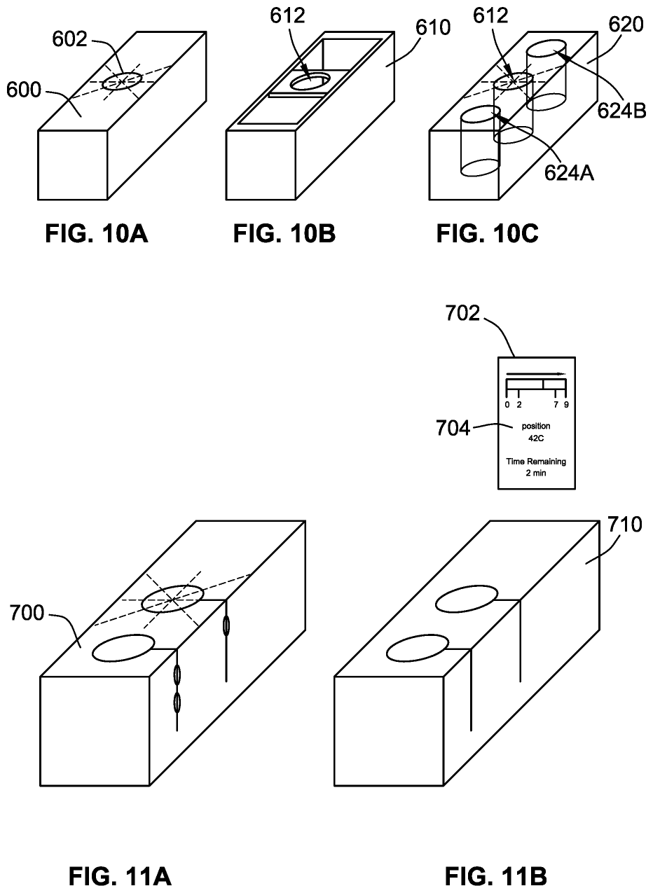

FIGS. 10A, 10B, and 10C depicts three different examples for temperature control of any of the devices 100, 200, 300, 400, and 500 disclosed herein. One example is a container 600, depicted in FIG. 10A. The container 600 is formed from a solid block of some material with sufficient thermal conductivity and heat capacity (e.g., aluminum), such that the container 600 can be heated to the appropriate single temperature (e.g., 42° C. or 60° C.) (for example under running tap water) at the beginning of a test, and maintain a temperature within an acceptable range of this starting temperature for the duration of the test. Alternatively, it can contain an embedded Peltier or resistance heating element, battery or outlet-powered, and a feedback controller that maintains a given temperature. The container 600 includes a slot 602 into which a device can be inserted. Heating or cooling the container 600 can then be used to adjust the temperature of the device.

A second example is an insulated container 610 depicted in FIG. 10B. The container 610 could be a vacuum container, could be made from Styrofoam, or could have other configurations allowing for insulating properties. The container 610 includes a slot 612 into which a device can be inserted. The interior of the container 610 is hollow, such that the container 610 can be filled with water at a desired temperature and maintained approximately at that temperature for a desired duration, to control the temperature of the device.

A third example is container 620 depicted in FIG. 10C. Container 620 contains a central slot 622 to hold the device, a hot reservoir 624A, and a cold reservoir 624B. The hot reservoir 624A can be filled with boiling water (e.g., 100° C.), and the cold reservoir 624B can be filled with a combination of cold water and ice cubes (e.g., 0° C.). Per the 1-D thermal conductivity relation

$$( Q = - {KA}_{c} \frac{dT}{dx} ) ,$$

any one or more of the conductivity (K), cross sectional area (A_{c}), and distance (dx) of thermal bridges (aluminum, copper, or other highly conducting material) between the device in the central slot 622, the hot reservoir 624A, and the cold reservoir 624B can be adjusted to program specific device temperatures. Highly thermally conductive material can be disposed around the central slot 622 and the hot and cold reservoirs 624A, 624B, but insulated from other components to prevent undesirable heat transfer. Thus, each of the containers 600, 610, and 620 form an isothermal heating block, to provide an isothermal condition to the device and sample being used. Any of the containers 600, 610, and 620 can easily be maintained at a desired temperature, which could be about 42° C. or about 60° C. depending on the assay being performed. Other temperatures can also be used.

FIG. 11A and 11B depicts two examples for timing control. Multi-step assays are generally complicated procedures where steps should be carried out at specific times. Thus, in some implementations, users can utilize a clock, watch, or separate timer to keep track of the time during the multi-step assay. In an example depicted in FIG. 11A, the device is inserted into a container 700 (which could be the same as or similar to containers 600, 610, and/or 620) that includes built-in timers and/or notification mechanisms (such as an alphanumeric display such as a liquid crystal display (LCD) screen, lights, light emitting diodes (LEDs), speakers, buzzers, or other human-perceptible notifications). The notification mechanisms can indicate when the desired temperature has been reached, when the device has been incubating for a desired time period, the current state of the reaction within the device, the desired state of the reaction within the device, when a given step has been completed, etc. Container 700 may also include a controller (such as a simple microprocessor) that is configured to operate any built-in timers and/or notification mechanisms.

In another example depicted in FIG. 11B, the device is inserted into a container 710 (which could be the same as or similar to containers 600, 610, and/or 620). A user device 702 (such as a cell phone, smart watch, tablet computer, laptop computer, desktop computer, etc.) can be used to monitor the timing and/or the temperature of the reaction in the device. The user device 702 could be connected (wireless or wired) to the container 710 to obtain information about the timing and temperature, and that information can be relayed to the user via a display screen 704 of the user device 702. In some implementations, the user device 702 can be used to prompt the user to perform various steps of the test (such as depositing substances, rotating the reaction container relative to the cap, etc.). The user device 702 can indicate to the user when the desired temperature has been reached, when the device has been incubating for a desired time period, the current state of the reaction within the device, the desired state of the reaction within the device, when a given step has been completed, etc. Thus, a timing mechanism can be utilized to guide the user through any external manipulation steps that are required to complete the assay.

Containers utilized for performing an assay (such as containers 600, 610, 620, 700, and/or 710) may also include a read-out device that can be coupled to the container, and/or built into the container. The read-out device is configured to indicate to a user the results of the assay. In some implementations, the read-out device is a fluorescent (or color) read-out device that includes a light source (such as an LED), a light filter, and a detector. The light source directs light at the sample, and any light that is emitted by the sample and/or reflects off of the sample will then pass through the filter and be detected by the detector. The result of the assay can be quantified based on properties (such as color, intensity, scattering angle, etc.) of the detected light.

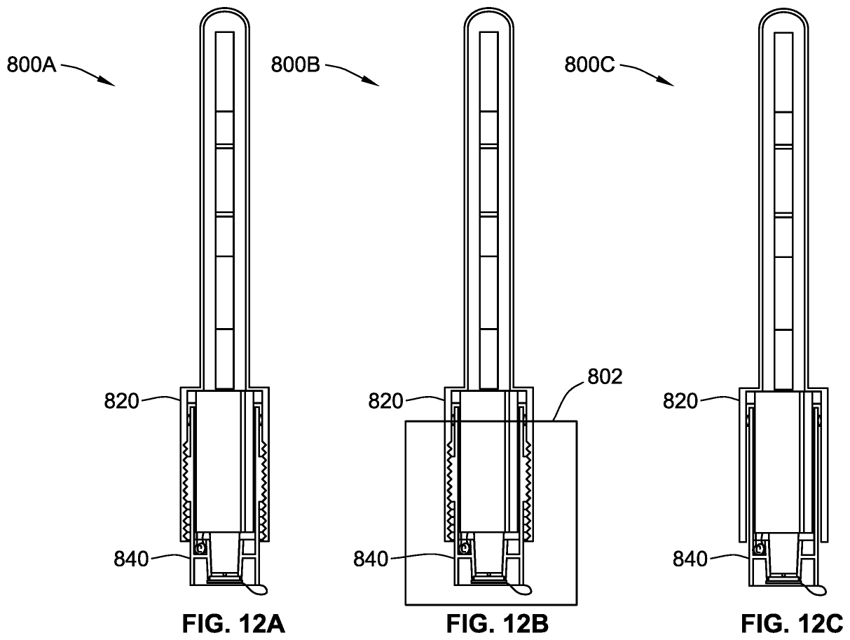

FIGS. 12A-12C depict different devices that utilize different advancement mechanisms to advance a reaction container 840 (which could be, for example, any of reaction containers 140, 240, 340, 420, or 520) toward a cap 820 (which could be, for example, any of caps 120, 220, or 310, or collection assemblies 410 or 510), within a device. In FIG. 12A, device 800A (which could be the same as or similar to any of devices 100, 200, 300, 400, or 500) allows the user to manually twist the reaction container 840 and/or the cap 820 in order to advance the reaction container 840 toward the cap 820. Thus, the advancement mechanism in FIG. 12A is rotation caused by the user. In FIG. 12B, device 800B (which could be the same as or similar to any of devices 100, 200, 300, 400, or 500) includes a stepper motor 802 that can be built into a container that holds the device 800B. The stepper motor 802 can automatically rotate the reaction container 840 relative to the cap 820 to advance the reaction container 840 toward the cap 820. Thus, the advancement mechanism in FIG. 12B includes the stepper motor 802.

In FIG. 12C, a device 800C (which could be the same as or similar to any of devices 100, 200, 300, 400, or 500) can be used. The device 800C does not include threads on the cap 820 and the reaction container 840, and thus the reaction container 840 is not rotatably moved toward the cap 820 to advance the test. Instead, the device 800C is constructed so that the reaction container 840 can be linearly moved toward the cap 820 to advance the test. The device 800C could include mechanisms (such as internal protrusions) that can temporarily halt the movement of the reaction container 840 toward the cap 820 when the reaction container 840 reaches a desired location, or can provide tactile feedback to the user (for example by applying a normal force against the user moving the reaction container 840) to indicate to the user that the user should temporarily stop moving the reaction container 840. These mechanisms can then be overcome to continue moving the reaction container 840 toward the cap 820. In some implementations, a user manually moves the reaction container 840 toward the cap 820. In these implementations, the underside of the reaction container 840 may include some sort of button or other structure to provide the user with a large surface onto which to place their finger and apply pressure to the reaction container 840. In other implementations, device 800C could be advanced using a stepper motor, such as stepper motor 802.

Generally, any of these advancement mechanisms can be multiplexed in a single large heating block, providing any of the time, temperature, or reaction advance steps described here. In addition, the block could also include a stepper motor (such as stepper motor 802) or other motor to automatically push the reaction container 840 toward the cap 820 as needed.

Generally, any combination of the heating mechanisms discussed with respect to FIGS. 10A-10C, the timing control mechanisms discussed with respect to FIGS. 11A and 11B, and the advancement mechanisms discussed with respect to FIGS. 12A-12C can be used with any of the described devices 100, 200, 300, 400, and/or 500.

Any of devices 100, 200, 300, 400, and 500 can be used to perform a variety of different assays or tests. In some implementations, devices 100-500 can be used to perform an amplification test to detect a target molecule. For example, devices 100-500 can be used to perform a polymerase chain reaction (PCR) test, a loop-mediated isothermal amplification (LAMP) test, a recombinase polymerase amplification (RPA) test, or other amplification tests. PCR tests generally involve changing the temperature of the sample, while LAMP tests and RPA tests are isothermal tests that do not involve changing the temperature of the sample. In these examples, an amplification reaction is performed on the sample, such that a target molecule in the sample is amplified (e.g., multiplied). The presence of that target molecule can then be detected.

In implementations using devices 100, 200, and 300, the lateral flow strips 102, 202, and 302 are used to detect the presence of the target molecule. Generally, the lateral flow strips 102, 202, and 302 include some substance that is configured to indicate the presence of the target molecule. The substance could be a capture reagent (such as a DNA oligonucleotide or an RNA oligonucleotide), a nanoparticle, or other substance. In some implementations, the lateral flow strips 102, 202, and 302 (or one or more portions thereof) may include substances that change color in the presence of the target molecule. This color change can be viewable through the tube 110, the tube 210, or the cap 310. In implementations using devices 400 and 500, the presence of the target molecule can result in the mixed liquids in the reaction chambers changing colors (e.g., a colorimetric reaction). This color change can be viewed through the walls of the reaction containers, which made be made from a transparent or semi-transparent material. Other types of tests or assays can also be performed that utilize different techniques to determine the result of the test or assay.

In some implementations, devices 100-500 can also be used for multiplexing. Multiplexing generally refers to performing multiple different assays or tests at the same time, to detect the presence of a target molecule in multiple different samples, or to amplify and detect multiple different target molecules in a sample or samples. In implementations using any of devices 100-300, the lateral flow strips 102-302 can include multiple physical locations with different capture reagents. The different capture reagents detect the presence of different target molecules. Thus, after the sample has undergone the amplification reaction and reached the lateral flow strip, any area of the lateral flow strip corresponding to a target molecule that was present in the sample and amplified can be detected. In some implementations, multiple different target molecules can be detected in the same sample using a single test. The substance or substances disposed within the devices 100-300 can be configured to amplify a single target molecule in the sample, or multiple target molecules in the sample.

In some implementations using devices 400 or 500, the multiple different reaction chambers can be used to simultaneously test for multiple different target molecules in the same sample. In these implementations, the same sample can be placed into each reaction chamber (e.g., the sample can be collected and a portion of the collected sample is placed into each reaction chamber), and each reaction chamber can have a substance configured to amplify a different target molecule. In other implementations using devices 400 or 500, the multiple different reaction chambers can be used to simultaneously test for the same target molecule in different samples. In these implementations, at least two different samples can be placed into their own reaction chamber, and each reaction chamber can have a substance configured to amplify the same target molecule. This substance may be the same substance for each reaction chamber containing a sample, or could be a different substance for each reaction chamber containing a sample, so long as the substances are configured to amplify the same target molecule. In further implementations using devices 400 or 500, the multiple different reaction chambers can be used to simultaneously test for different target molecules in different samples. In these implementations, at least two reaction chambers contain the same sample (e.g., portions of a sample collected from one source), and a third reaction chamber contains a different sample. The two reaction chambers containing the same sample can contain different substances to amplify different target molecules, while the third reaction chamber can contain any desired substance to amplify any desired target molecule.

A number of different samples can be tested using devices 100-500, such as blood, serum, plasma, urine, semen, mucus, synovial fluid, bile fluid, cerebrospinal fluid, mucosal secretion, effusion, sweat, saliva, etc. The sample could also be a biopsy sample, a tumor sample, or a tissue sample. The sample could further be any combination or mixture of the above-mentioned samples. The target molecule in the sample can be a target protein, a target nucleic acid, or other target molecules. The target nucleic acid can be any desired nucleic acid. Further, the target nucleic acid can include naturally occurring or synthetic nucleic acids. A naturally occurring nucleic acid includes a nucleic acid isolated and/or purified from a natural source.

In some implementations, the target nucleic acid is DNA, e.g., a target DNA. Exemplary target DNAs include, but are not limited to, genomic DNA, viral DNA, cDNA, single-stranded DNA, double-stranded DNA, circular DNA, etc. In some implementations, the target nucleic acid is an RNA, e.g., a target RNA. Generally, the RNA can be any known type of RNA. In some implementations, the target RNA is messenger RNA, ribosomal RNA, Signal recognition particle RNA, Transfer RNA, Transfer-messenger RNA, Small nuclear RNA, Small nucleolar RNA, SmY RNA, Small Cajal body-specific RNA, Guide RNA, Ribonuclease P, Ribonuclease MRP, Y RNA, Telomerase RNA Component, Spliced Leader RNA, Antisense RNA, Cis-natural antisense transcript, CRISPR RNA, Long noncoding RNA, MicroRNA, Piwi-interacting RNA, Small interfering RNA, Short hairpin RNA, Trans-acting siRNA, Repeat associated siRNA, 7SK RNA, Enhancer RNA, Parasitic RNAs, Type, Retrotransposon, Viral genome, Viroid, Satellite RNA, or Vault RNA.

In some implementations, the target RNA can be a viral RNA. As used herein, the term “RNA virus” refers to a virus comprising an RNA genome. In some implementations, the RNA virus is a double-stranded RNA virus, a positive-sense RNA virus, a negative-sense RNA virus, or a reverse transcribing virus (e.g., retrovirus).

In some implementations, the RNA virus is a Group III (i.e., double stranded RNA (dsRNA)) virus. In some implementations, the Group III RNA virus belongs to a viral family selected from the group consisting of: Amalgaviridae, Birnaviridae, Chrysoviridae, Cystoviridae, Endornaviridae, Hypoviridae, Megabirnaviridae, Partitiviridae, Picobirnaviridae, Reoviridae (e.g., Rotavirus), Totiviridae, Quadriviridae. In some implementations, the Group III RNA virus belongs to the Genus Botybirnavirus. In some implementations, the Group III RNA virus is an unassigned species selected from the group consisting of: Botrytis porri RNA virus 1, Circulifer tenellus virus 1, Colletotrichum camelliae filamentous virus 1, Cucurbit yellows associated virus, Sclerotinia sclerotiorum debilitation-associated virus, and Spissistilus festinus virus 1.

In some implementations, the RNA virus is a Group IV (i.e., positive-sense single stranded (ssRNA)) virus. In some implementations, the Group IV RNA virus belongs to a viral order selected from the group consisting of: Nidovirales, Picornavirales, and Tymovirales. In some implementations, the Group IV RNA virus belongs to a viral family selected from the group consisting of: Arteriviridae, Coronaviridae (e.g., Coronavirus, SARS-CoV), Mesoniviridae, Roniviridae, Dicistroviridae, Iflaviridae, Marnaviridae, Picornaviridae (e.g., Poliovirus, Rhinovirus (a common cold virus), Hepatitis A virus), Secoviridae (e.g., sub Comovirinae), Alphaflexiviridae, Betaflexiviridae, Gammaflexiviridae, Tymoviridae, Alphatetraviridae, Alvernaviridae, Astroviridae, Barnaviridae, Benyviridae, Bromoviridae, Caliciviridae (e.g., Norwalk virus), Carmotetraviridae, Closteroviridae, Flaviviridae (e.g., Yellow fever virus, West Nile virus, Hepatitis C virus, Dengue fever virus, Zika virus), Fusariviridae, Hepeviridae, Hypoviridae, Leviviridae, Luteoviridae (e.g., Barley yellow dwarf virus), Polycipiviridae, Narnaviridae, Nodaviridae, Permutotetraviridae, Potyviridae, Sarthroviridae, Statovirus, Togaviridae (e.g., Rubella virus, Ross River virus, Sindbis virus, Chikungunya virus), Tombusviridae, and Virgaviridae. In some implementations, the Group IV RNA virus belongs to a viral genus selected from the group consisting of: Bacillariornavirus, Dicipivirus, Labyrnavirus, Sequiviridae, Blunervirus, Cilevirus, Higrevirus, Idaeovirus, Negevirus, Ourmiavirus, Polemovirus, Sinaivirus, and Sobemovirus. In some implementations, the Group IV RNA virus is an unassigned species selected from the group consisting of: Acyrthosiphon pisum virus, Bastrovirus, Blackford virus, Blueberry necrotic ring blotch virus, Cadicistrovirus, Chara australis virus, Extra small virus, Goji berry chlorosis virus, Hepelivirus, Jingmen tick virus, Le Blanc virus, Nedicistrovirus, Nesidiocoris tenuis virus 1, Niflavirus, Nylanderia fulva virus 1, Orsay virus, Osedax japonicus RNA virus 1, Picalivirus, Plasmopara halstedii virus, Rosellinia necatrix fusarivirus 1, Santeuil virus, Secalivirus, Solenopsis invicta virus 3, Wuhan large pig roundworm virus. In some implementations, the Group IV RNA virus is a satellite virus selected from the group consisting of: Family Sarthroviridae, Genus Albetovirus, Genus Aumaivirus, Genus Papanivirus, Genus Virtovirus, and Chronic bee paralysis virus.

In some implementations, the RNA virus is a Group V (i.e., negative-sense ssRNA) virus. In some implementations, the Group V RNA virus belongs to a viral phylum or subphylum selected from the group consisting of: Negarnaviricota, Haploviricotina, and Polyploviricotina. In some implementations, the Group V RNA virus belongs to a viral class selected from the group consisting of: Chunqiuviricetes, Ellioviricetes, Insthoviricetes, Milneviricetes, Monjiviricetes, and Yunchangviricetes. In some implementations, the Group V RNA virus belongs to a viral order selected from the group consisting of: Articulavirales, Bunyavirales, Goujianvirales, Jingchuvirales, Mononegavirales, Muvirales, and Serpentovirales. In some implementations, the Group V RNA virus belongs to a viral family selected from the group consisting of: Amnoonviridae (e.g., Taastrup virus), Arenaviridae (e.g., Lassa virus), Aspiviridae, Bornaviridae (e.g., Borna disease virus), Chuviridae, Cruliviridae, Feraviridae, Filoviridae (e.g., Ebola virus, Marburg virus), Fimoviridae, Hantaviridae, Jonviridae, Mymonaviridae, Nairoviridae, Nyamiviridae, Orthomyxoviridae (e.g., Influenza viruses), Paramyxoviridae (e.g., Measles virus, Mumps virus, Nipah virus, Hendra virus, and NDV), Peribunyaviridae, Phasmaviridae, Phenuiviridae, Pneumoviridae (e.g., RSV and Metapneumovirus), Qinviridae, Rhabdoviridae (e.g., Rabies virus), Sunviridae, Tospoviridae, and Yueviridae. In some implementations, the Group V RNA virus belongs to a viral genus selected from the group consisting of: Anphevirus, Arlivirus, Chengtivirus, Crustavirus, Tilapineviridae, Wastrivirus, and Deltavirus (e.g., Hepatitis D virus).

In some implementations, the RNA virus is a Group VI RNA virus, which comprise a virally encoded reverse transcriptase. In some implementations, the Group VI RNA virus belongs to the viral order Ortervirales. In some implementations, the Group VI RNA virus belongs to a viral family or subfamily selected from the group consisting of: Belpaoviridae, Caulimoviridae, Metaviridae, Pseudoviridae, Retroviridae (e.g., Retroviruses, e.g. HIV), Orthoretrovirinae, and Spumaretrovirinae. In some implementations, the Group VI RNA virus belongs to a viral genus selected from the group consisting of: Alpharetrovirus (e.g., Avian leukosis virus; Rous sarcoma virus), Betaretrovirus (e.g., Mouse mammary tumour virus), Bovispumavirus (e.g., Bovine foamy virus), Deltaretrovirus (e.g., Bovine leukemia virus; Human T-lymphotropic virus), Epsilonretrovirus (e.g., Walleye dermal sarcoma virus), Equispumavirus (e.g., Equine foamy virus), Felispumavirus (e.g., Feline foamy virus), Gammaretrovirus (e.g., Murine leukemia virus; Feline leukemia virus), Lentivirus (e.g., Human immunodeficiency virus 1; Simian immunodeficiency virus; Feline immunodeficiency virus), Prosimiispumavirus (e.g., Brown greater galago prosimian foamy virus), and Simiispumavirus (e.g., Eastern chimpanzee simian foamy virus).

In some implementations, the RNA virus is selected from influenza virus, human immunodeficiency virus (HIV), and severe acute respiratory syndrome coronavirus 2 (SARS-CoV-2). In some implementations, the RNA virus is influenza virus. In some implementations, the RNA virus is immunodeficiency virus (HIV). In some implementations, the RNA virus is severe acute respiratory syndrome coronavirus 2 (SARS-CoV-2).

In some implementations, the viral RNA is an RNA produced by a virus with a DNA genome, i.e., a DNA virus. As a non-limiting example the DNA virus is a Group I (dsDNA) virus, a Group II (ssDNA) virus, or a Group VII (dsDNA-RT) virus.

In some implementations, at least one member of the plurality of target nucleic acids is single-stranded. In some implementations, at least one member of the plurality of target nucleic acids is double-stranded. In some implementations, at least one member of the plurality of target nucleic acids is RNA. In some implementations, at least one member of the plurality of target nucleic acids is DNA. In some implementations, at least one member of the plurality of target nucleic acids is a viral nucleic acid. In some implementations, at least one member of the plurality of target nucleic acids is a first viral nucleic acid and at least one member of the plurality of target nucleic acids is a second viral nucleic acid. For example, the first and second viral nucleic acids are from different viruses. In some implementations, at least one member of the plurality of target nucleic acids is a viral RNA. In some implementations, at least one member of the plurality of target nucleic acids is a viral DNA.

In some implementations, the target nucleic acid includes bacterial DNA, bacterial RNA, viral DNA, viral RNA, fungal DNA, fungal RNA, eukaryotic DNA, eukaryotic RNA, prokaryotic DNA, prokaryotic RNA, or any combination thereof.

In some implementations, multiple devices can be used simultaneously in an array to test multiple different samples. The devices can be arranged so that they can all be simultaneously physically manipulated, for example to advance the reaction (devices 100-300) or too couple the collection assembly to the reaction chamber (devices 400 and 500).

In some implementations, devices 100-500 can be single-use devices. In these implementations, devices 100-500 can include a one-way closure mechanism. The one-way closure mechanism allows the reaction chamber to be coupled to the rest of the device (e.g., the tube and/or cap, or the collection assemblies) once the samples are collected and deposited into the reaction chamber. The one-way closure mechanism then prevents the devices from being disassembled after the assay or test has been performed, so that amplified target molecules in the devices do not pose any contamination risk. In other implementations however, devices 100-500 could be reusable.

The description of implementations of the disclosure is not intended to be exhaustive or to limit the disclosure to the precise form disclosed. While specific implementations of, and examples for, the disclosure are described herein for illustrative purposes, various equivalent modifications are possible within the scope of the disclosure, as those skilled in the relevant art will recognize. For example, while method steps or functions are presented in a given order, alternative implementations may perform functions in a different order, or functions may be performed substantially concurrently. The teachings of the disclosure provided herein can be applied to other procedures or methods as appropriate. The various implementations described herein can be combined to provide further implementations. Aspects of the disclosure can be modified, if necessary, to employ the compositions, functions and concepts of the above references and application to provide yet further implementations of the disclosure. These and other changes can be made to the disclosure in light of the detailed description. All such modifications are intended to be included within the scope of the appended claims.

What is claimed is:

1. A device for performing a multi-step assay, the device comprising:
a tube including a lateral flow strip disposed therein;
a cap coupled to the tube and including a hollow interior defined at least partially therethrough;
an insert configured to be at least partially received within the hollow interior of the cap; and
a reaction container including a cavity configured to store one or more fluids therein, the reaction container being rotatably coupled to the cap such that rotation of the cap relative to the reaction container causes (i) mixing of the one or more fluids and (ii) at least a portion of the mixed fluids to be delivered from the reaction container to the lateral flow strip via the insert.

2. The device of claim 1, wherein the cap is coupled to the reaction container via a threaded connection.

3. The device of claim 1, wherein the tube and the cap are unitary or monolithic.

4. The device of claim 1, wherein the reaction container includes a plurality of wells.

5. The device of claim 4, wherein the plurality of wells of the reaction container include a first well configured to store a first reagent therein, a second well configured to store a second reagent therein, and a third well configured to store a buffer therein.

6. The device of claim 5, wherein the first reagent is a recombinase polymerase amplification (RPA) reagent, the second reagent is a sodium dodecyl sulfate (SDS) reagent, and the buffer is an exonuclease reaction buffer.

7. The device of claim 1, wherein the reaction container includes an O-ring.

8. The device of claim 5, wherein the reaction container includes a seal covering a first end of the third well, and a removable cap covering a second end of the third well.

9. The device of claim 8, wherein the insert includes a body, a displacing bump extending from the body, a brush extending from the body, and an aperture defined through the body.

10. The device of claim 9, wherein the brush of the insert aids in mixing the first reagent stored in the first well with the second reagent stored in the second well responsive to the reaction container being rotated relative to the cap toward a first position to cause corresponding rotation of the insert.

11. The device of claim 10, wherein the displacing bump is configured to break the seal of the third well to mix the buffer with the mixed first reagent and second reagent responsive to the reaction container being rotated relative to the cap from the first position toward a second position.

12. The device of claim 11, wherein the aperture of the body of the insert is configured to deliver the mixed first reagent, second reagent, and buffer from the reaction chamber to the lateral flow strip responsive to the reaction container being rotated relative to the cap from the second position toward a third position.

13. The device of claim 1, wherein the cap includes a plurality of slots configured to engage the insert, such that the insert is rotationally locked to the cap.

14. The device of claim 1, wherein the tube is coupled to the cap via a threaded connection.

15. The device of claim 1, wherein the reaction container is coupled to the cap via a threaded connection.

16. The device of claim 1, wherein the reaction container is configured to store a first reagent therein and the insert includes a blister pack configured to store a buffer therein.

17. The device of claim 16, wherein the first reagent is a recombinase polymerase amplification (RPA) reagent and the buffer is an exonuclease reaction buffer.

18. The device of claim 16, wherein the reaction container includes a protrusion configured to engage the blister pack of the insert to cause mixing of the first reagent and the buffer responsive to the reaction container being rotated relative to the cap toward a first position.

19. The device of claim 18, wherein the blister pack and the protrusion are generally conical.

20. The device of claim 19, wherein the protrusion includes one or more vanes extending from a surface of the protrusion, the one or more vanes being configured to aid in causing mixing of the first reagent and the buffer responsive to rotation of the cap towards the first position.

21. The device of claim 20, wherein the one or more vanes are arranged in a helical pattern, a semi-helical pattern, a vertical pattern, a horizontal pattern, a diagonal pattern, or any combination thereof.

22. A device for performing a multi-step assay, the device comprising:
a cap including a hollow interior defined at least partially therethrough;
a lateral flow strip;
a plunger assembly configured to be received within the hollow interior of the cap;
a reagent insert including a plurality of apertures, a slot, and a seal, the slot being configured to receive a portion of the lateral flow strip therein, the seal being positioned such that the plurality of apertures can store a first fluid and a second fluid, or both therein; and
a reaction container including an internal cavity for storing a third fluid, the internal cavity being configured to receive a portion of the reagent insert therein, the reaction container being coupled to the cap such that (i) rotation of the cap relative to the reaction container towards a first position causes the plunger assembly to mix the first fluid and the third fluid, (ii) rotation of the cap relative to the reaction container from the first position towards a second position causes the plunger assembly to mix the first fluid, the second fluid, and the third fluid, and (iii) rotation of the cap relative to the reaction container from the second position towards a third position causes at least a portion of the lateral flow strip to be disposed within the mixed first fluid, second fluid, and third fluid.

23. The device of claim 22, wherein the cap is coupled to the reaction container via a threaded connection.

24. The device of claim 22, wherein the plunger assembly includes a primary plunger having a first length and a first tip, and a secondary plunger having a second length and a second tip, the first length being greater than the second length.

25. The device of claim 24, wherein the primary plunger includes one or more notches configured to cause at least a portion of the primary plunger to buckle responsive to rotation of the cap towards the second position.

26. The device of claim 24, wherein the plurality of apertures of the reagent insert include a primary aperture and a secondary aperture, the primary aperture being configured to receive a portion of the primary plunger and store the first fluid therein, the secondary aperture being configured to receive a portion of the secondary plunger therein and store the second fluid therein.

27. The device of claim 25, wherein the primary aperture has a first aperture diameter and the secondary aperture has a second aperture diameter that is greater than the first aperture diameter.

28. The device of claim 27, wherein the primary plunger has a first plunger diameter and the secondary plunger has a second plunger diameter that is greater than the first plunger diameter.

29. The device of claim 28, wherein the second plunger diameter of the secondary plunger is greater than the first plunger diameter of the primary plunger and the first aperture diameter of the primary aperture.

30. The device of claim 26, wherein (i) rotation of the cap relative to the reaction container towards the first position causes the first tip of the primary plunger to pierce the seal to mix the first fluid and the third fluid, and (ii) rotation of the cap relative to the reaction container from the first position towards the second position causes the second tip of the secondary plunger to pierce the seal to mix the first fluid, the second fluid, and the third fluid.

31. The device of claim 22, wherein the cap is transparent.

32. The device of claim 22, wherein the first fluid is a first reagent, the second fluid is a buffer, and the third fluid is a second reagent.

33. A device for performing one or more tests on one or more samples, the device comprising:
a collection assembly including a handle and a plurality of collection swabs extending from the handle; and
a reaction container including a plurality of reaction chambers, each of the plurality of reaction chambers being associated with a corresponding one of the plurality of collection swabs,
wherein in response to a configuration of the device moving from an unassembled configuration to an assembled configuration, the collection assembly is coupled to the reaction container, and each of the plurality of reaction chambers at least partially houses the corresponding one of the plurality of collection swabs therein.

34. The device of claim 33, wherein the plurality of collection swabs is arranged linearly, and the plurality of reaction chambers is arranged linearly.

35. The device of claim 34, wherein the plurality of collection swabs includes at least a first collection swab, a second collection swab, and a third collection swab, the third collection swab being positioned between the first collection swab and the second collection swab along a linear axis.

36. The device of claim 34, wherein the plurality of reaction chambers includes at least a first reaction chamber, a second reaction chamber, and a third reaction chamber, the third reaction chamber being positioned between the first reaction chamber and the second reaction chamber along a linear axis.

37. The device of claim 33, wherein the plurality of collection swabs is arranged circularly, and the plurality of reaction chambers is arranged circularly.

38. The device of claim 37, wherein the reaction container has a circular cross-section, and wherein each of the plurality of reaction chambers occupies a portion of the reaction chamber that is between about 100° and about 120° of a circumference of the reaction chamber.

39. The device of claim 33, wherein each of the plurality of collection swabs includes one or more apertures defined therein.

40. The device of claim 33, wherein each of the plurality of collection swabs is configured to contain a sample to be tested, and wherein each of the plurality of reaction chambers includes at least one substance for performing a test.

41. The device of claim 40, further comprising a plurality of mixing mechanisms, each mixing mechanism being configured to aid in mixing the at least one substance in a corresponding one of the plurality of reaction chambers and the sample contained by the corresponding collection swab associated with the corresponding one of the plurality of reaction chambers.

42. A method for performing a multi-step assay, the method comprising:
depositing one or more reagents and a buffer in a reaction container;
coupling the reaction chamber to a cap and an insert; and
causing the cap to move towards a first position to cause mixing of the one or more reagents, the buffer, or both.

43. The method of claim 42, wherein the causing the cap to move towards the first position includes rotating the cap relative to the reaction container.

44. The method of claim 42, wherein the one or more reagents includes a first reagent and a second reagent, and the depositing the one or more reagents in a reaction container includes depositing the first reagent in a first well of the reaction container, the second reagent in a second well of the reaction container, and the buffer in a cavity of the reaction container.

45. The method of claim 44, wherein the first reagent is recombinase polymerase amplification (RCA) reagent and the second reagent is a sodium dodecyl sulfate (SDS) reagent.

46. The method of claim 44, wherein the causing the cap to move towards the first position causes mixing of the first reagent and the second reagent.

47. The method of claim 46, further comprising incubating the mixed first reagent and second reagent for a first predetermined time at a first predetermined temperature.

48. The method of claim 47, wherein the first predetermined time is about 5 minutes.

49. The method of claim 47, wherein the first predetermined temperature is about 42° C.

50. The method of claim 47, further comprising:
causing the cap to move from the first position towards a second position to cause mixing of the first reagent, the second reagent, and the buffer.

51. The method of claim 50, wherein the causing the cap to move toward the second position includes rotating the cap relative to the reaction container.

52. The method of claim 50, further comprising:
incubating the mixed first reagent, second reagent, and buffer for a second predetermined time at a second predetermined temperature.

53. The method of claim 52, wherein the second predetermined time is about 1 minute.

54. The method of claim 52, wherein the second predetermined temperature is between about 20° C. and about 22° C.

55. The method of claim 52, further comprising:
causing the cap to move from the second position towards a third position to cause the mixed first reagent, second reagent, and buffer to be transported from the reaction container towards a test strip.

56. The method of claim 55, wherein the causing the cap to move toward the third position includes rotating the cap relative to the reaction container.

57. The method of claim 55, further comprising:
incubating the mixed first reagent, second reagent, and buffer subsequent to the mixed first reagent, second reagent, and buffer being transported towards the lateral flow strip for a third predetermined time at a third predetermined temperature.

58. The method of claim 57, wherein the third predetermined time is about 1 minute.

59. The method of claim 57, wherein the third predetermined temperature is between about 20° C. and about 22° C.

60. The method of claim 42, wherein the test strip is a lateral flow device.

61. The method of claim 42, wherein the depositing the one or more reagents in the reaction container includes depositing a first reagent in the reaction container and depositing the buffer in the insert.

62. The method of claim 61, wherein the causing the cap to move towards the first position includes rotating the cap relative to the reaction container to cause mixing the first reagent and the buffer.

63. The method of claim 42, wherein the cap is coupled to a plunger assembly including a primary plunger and a secondary plunger.

64. The method of claim 63, wherein the one or more reagents includes a first reagent and the insert includes a seal, a primary aperture for storing a second reagent, and a secondary aperture for storing a buffer.

65. The method of claim 64, wherein the causing the cap to move towards the first position includes causing the primary plunger of the plunger assembly to break a first portion of the seal of the insert to deliver the second reagent to the reaction container.

66. The method of claim 65, further comprising mixing the first reagent and the second reagent in the reaction container.

67. The method of claim 66, further comprising incubating the first reagent and second reagent subsequent to the mixing the first reagent and the second reagent in the reaction container.

68. The method of claim 65, further comprising causing the cap to move from the first position towards a second position to cause the secondary plunger of the plunger assembly to break a second portion of the seal of the insert to deliver the buffer to the reaction container.

69. The method of claim 68, further comprising mixing the first reagent, the second reagent, and the buffer in the reaction container.

70. The method of claim 69, further comprising incubating the mixed first reagent, second reagent, and buffer.

71. The method of claim 70, further comprising causing at least a portion of a test strip to be disposed within the mixed first reagent, second reagent, and buffer.

72. The method of claim 71, further comprising causing the cap to move from the second position towards a third position to cause the test strip to be at least partially disposed within the mixed first reagent, second reagent, and buffer in the reaction container.

73. The method of claim 42, wherein the causing the cap to move towards the first position includes prompting a user to rotate the cap relative to the reaction container via a user device.

74. The method of claim 42, wherein the cap, the insert, and the reaction chamber are at least partially disposed within a receptacle of a testing container.

75. The method of claim 74, wherein the causing the cap to move towards the first position includes causing one or more motors of the testing container to rotate the cap relative to the reaction container.

76. The method of claim 74, wherein the testing container includes one or more heating elements and a control system for heating the reaction chamber to a predetermined temperature.
